(19) United States
(12) Patent Application Publication
(10) Pub. No.: US 2023/0041418 A1
(43) Pub. Date: Feb. 9, 2023

(54) METHOD

(71) Applicant: Oxford Nanopore Technologies PLC, Oxford (GB)

(72) Inventors: Clive Gavin Brown, Oxford (GB); Andrew John Heron, Oxford (GB); James Anthony Clarke, Oxford (GB); Paul Richard Moody, Oxford (GB); Aaron Luke Acton, Oxford (GB); Jason Robert Hyde, Oxford (GB)

(73) Assignee: Oxford Nanopore Technologies PLC, Oxford (GB)

(21) Appl. No.: 17/781,820

(22) PCT Filed: Dec. 3, 2020
(86) PCT No.: PCT/GB2020/053104

§ 371 Date: Jun. 2, 2022

(30) Foreign Application Priority Data
Dec. 4, 2019 (GB) 1917742.7

(51) Int. Cl. C12Q 1/6825; B01L 3/00

(52) U.S. Cl. CPC C12Q 1/6825; B01L 3/502761; B01L 2300/161; B01L 2300/0645; B01L 2200/16; B01L 2200/0647

(57) ABSTRACT

Provided herein is a method of concentrating a tethering complex in a region of an amphiphilic layer, such as a lipid membrane. Also provided herein are methods of assembling a tethering complex; methods of concentrating an analyte in the region of a detector; amphiphilic layers; and arrays and devices for use in the disclosed methods.

### RELATED APPLICATIONS

This application is a national stage filing under 35 U.S.C. § 371 of international application number PCT/GB2020/053104, filed Dec. 3, 2020, which claims the benefit of Great Britain application number GB 1917742.7, filed Dec. 4, 2019, each of which are herein incorporated by reference in their entirety.

### REFERENCE TO A SEQUENCE LISTING SUBMITTED AS A TEST FILE VIA EFS-WEB

The instant application contains a Sequence Listing which has been submitted in ASCII format via EFS-Web and is hereby incorporated by reference in its entirety. Said ASCII copy, created on Jun. 2, 2022, is named 0036670129US00-SEQ-KZM and is 38,246 bytes in size.

### FIELD

The present disclosure relates to methods of concentrating a tethering complex in a region of an amphiphilic layer, such as a lipid membrane. The disclosure also relates to methods of assembling a tethering complex; to methods of concentrating an analyte in the region of a detector; to amphiphilic layers; and to arrays and devices for use in the disclosed methods.

### BACKGROUND

The characterisation of biological molecules is of increasing importance in biomedical and biotechnological applications.

For example, sequencing of nucleic acids allows the study of genomes and the proteins they encode and, for example, allows correlation between nucleic acid mutations and observable phenomena such as disease indications. Sequencing can be used in evolutionary biology to study the relationship between organisms. Metagenomics involves identifying organisms present in samples, for example microbes in a microbiome, with nucleic acid sequencing allowing the identification of such organisms. In medicine, genetic testing of a subject may highlight the risk of genetic disease or allow the selection of optimum therapies to treat medical conditions. DNA sequencing is also a key technology in forensic science.

Characterisation of other analytes such as polypeptides is also very important. For example, knowledge of a protein sequence can allow structure-activity relationships to be established and has implications in rational drug development strategies for developing ligands for specific receptors. Identification of post-translational modifications is also key to understanding the functional properties of many proteins. For example, typically 30-50% of protein species are phosphorylated in eukaryotes. Some proteins may have multiple phosphorylation sites, serving to activate or inactivate a protein, promote its degradation, or modulate interactions with protein partners.

Analytes including nucleic acids and polypeptides can be characterised using many known technologies. Single molecule techniques have proven to be particularly attractive due to their high fidelity, avoidance of amplification bias, and the possibility of achieving extremely long read lengths. Single molecule sequencing of polynucleotides and polypeptides can also provide information on the presence of characteristics such as base modifications, oxidation, reduction, decarboxylation, deamination, post-translational modifications and more.

One attractive method of single molecule analyte characterisation is nanopore sensing. Nanopore sensing is an approach to analyte detection and characterization that relies on the observation of individual binding or interaction events between the analyte molecules and an ion conducting channel. Nanopore sensors can be created by placing a single pore of nanometre dimensions in an electrically insulating membrane and measuring voltage-driven ion currents through the pore in the presence of analyte molecules. The presence of an analyte inside or near the nanopore will alter the ionic flow through the pore, resulting in altered ionic or electric currents being measured over the channel. The identity of an analyte is revealed through its distinctive current signature, notably the duration and extent of current blocks and the variance of current levels during its interaction time with the pore. Nanopore sensing has the potential to allow rapid and cheap polynucleotide sequencing, providing single molecule sequence reads of polynucleotides of tens to tens of thousands bases length. Nanopore sensing also has the potential to allow rapid and cheap characterisation of other technologically important analytes including polypeptides.

For nanopore applications, efficient capture of analyte is required. For instance, in DNA sequencing it is important that a new analyte is captured by the pore as soon as the previous one has been processed. Similar considerations apply in the nanopore characterisation of other analytes such as polypeptides.

To address this problem, it is known to use a hydrophobic tether (also known as an anchor) to couple the analyte to the membrane in which the nanopore is located. By coupling the analyte to the membrane, the local concentration of the analyte near the nanopore is increased relative to the bulk solution. This leads to improvements in the efficiency at which the analyte can be characterised using a nanopore.

Whilst such coupling methods have proven to be useful in facilitating the characterisation of analytes such as polynucleotides and polypeptides, some technical problems remain. One issue is that the tethers used to couple the analyte to the membrane are typically unselective as regards the locations they are able to access; e.g. they are typically unselective regarding the chemical environment in which the amphiphilic molecules to which the tethers bind and which are used to produce the membrane are found. This leads to a loss of efficiency, as if such amphiphilic molecules are not in the vicinity of the nanopore, the analyte molecules coupled thereto may not be available to the nanopore for characterisation. Accordingly, further methods of concentrating analytes in the region of an nanopore are required.

### SUMMARY

The disclosure relates to methods of concentrating a tethering complex in a region of an amphiphilic layer such as a membrane. The amphiphilic layer comprises a plurality of amphiphilic molecules and a detector such as an nanopore. Whilst the disclosure provides nanopores as exemplary detectors, the methods provided herein are amenable to detectors including (i) a zero-mode waveguide, (ii) a field-effect transistor, optionally a nanowire field-effect transistor; (iii) an AFM tip; (iv) a nanotube, optionally a carbon nanotube and (v) a nanopore.

The tethering complex can be used to attach to an analyte to enable the analyte to be concentrated in the region of the amphiphilic layer in which the tethering complex is concentrated. By concentrating the tethering complex in the region of the amphiphilic layer comprising the nanopore, the analyte to be detected using the nanopore can be concentrated in the vicinity of the nanopore.

The tethering complex for use in the disclosed methods comprises one or more hydrophilic components connected by a hydrophobic linker. A hydrophilic component may for example be a component which can be attached to an analyte to enable the localisation of the analyte and thus promote its characterisation.

The disclosed methods comprise contacting the tethering complex, or one or more components thereof, with the amphiphilic molecules. The amphiphilic layer comprises a first region comprising the detector, and a second region, wherein the first region differs chemically and/or physically from the second region, and wherein the tethering complex preferentially localises to the first region relative to the second region. In this way, the tethering complex, and thus analyte molecules attached to the tethering complex, are concentrated in the vicinity of the nanopore.

Accordingly, the present disclosure provides a method of concentrating a tethering complex in a region of an amphiphilic layer, said amphiphilic layer comprising a plurality of amphiphilic molecules and a detector, wherein the tethering complex comprises one or more hydrophilic components connected by a hydrophobic linker;

the method comprising contacting the tethering complex or one or more components thereof with said plurality of amphiphilic molecules;

and wherein the amphiphilic layer comprises a first region comprising the detector, and a second region, wherein the first region differs chemically and/or physically from the second region, and wherein the tethering complex preferentially localises to the first region relative to the second region; thereby concentrating the tethering complex in the first region of the amphiphilic layer.

In some embodiments the tethering complex comprises a first hydrophilic component connected to a second hydrophilic component by a hydrophobic linker.

In some embodiments the first region is a multi-layered region of the amphiphilic layer.

In some embodiments, the first region and the second region both comprise the same type of amphiphilic molecules. In some embodiments the first region comprises a first composition of amphiphilic molecules and the amphiphilic second region comprises a second composition of amphiphilic molecules, and the first composition differs from the second composition.

In some embodiments, the first region and the second region of the amphiphilic layer respectively correspond to first and second areas of a substrate, wherein first area of the substrate differs chemically and/or physically from the second area. In some embodiments the first area corresponds to an aperture in a substrate and the second area corresponds to an optionally coated portion of the substrate. In some embodiments the first region corresponds to the interfacial surface area between a first droplet and a second droplet pair, wherein the first and second droplets each have an amphiphilic coating; and the second region corresponds to the surface area of the portion of the first droplet which does not interface with a second droplet. In some embodiments the first region and the second region are phase separated regions of the amphiphilic layer.

In some embodiments, the tethering complex is assembled as described herein.

Also provided herein is a method for assembling a tethering complex in an amphiphilic layer, wherein the tethering complex comprises one or more hydrophilic components connected by a hydrophobic linker; the method comprising contacting the tethering complex or one or more components thereof with a plurality of amphiphilic molecules and subsequently forming the amphiphilic layer.

Also provided is a method for assembling a tethering complex in an amphiphilic layer, wherein the tethering complex comprises one or more hydrophilic components connected by a hydrophobic linker; the method comprising (i) forming the amphiphilic layer from a plurality of amphiphilic molecules; and (ii) contacting the amphiphilic layer with the tethering complex or one or more components thereof

In some embodiments, the above methods comprise (i) contacting the hydrophobic linker with the amphiphilic molecules or the amphiphilic layer; wherein the hydrophobic linker is not attached to at least one of the one or more hydrophilic components when the hydrophobic linker is contacted with the amphiphilic molecules or amphiphilic layer and (ii) attaching at least one of the one or more hydrophilic components to the hydrophobic linker once the amphiphilic layer has been formed, thereby forming the tethering complex.

Also provided herein is a method for assembling a tethering complex in an amphiphilic layer, wherein the tethering complex comprises a first hydrophilic component connected to a second hydrophilic component by a hydrophobic linker, the method comprising contacting the tethering complex or one or more components thereof with a plurality of amphiphilic molecules and subsequently forming the amphiphilic layer.

In some embodiments the method comprises (i) contacting the hydrophobic linker with the plurality of amphiphilic molecules; and (ii) forming the amphiphilic layer. In some embodiments the hydrophobic linker is attached to at least one of the first hydrophilic component and/or the second hydrophilic component.

In some embodiments the hydrophobic linker is not attached to the first hydrophilic component and/or the second hydrophilic component when the hydrophobic linker is contacted with the amphiphilic molecules, and the method further comprises attaching the first hydrophilic component and/or the second hydrophilic component to the hydrophobic linker once the amphiphilic layer has been formed thereby forming the tethering complex.

In some embodiments, the method comprises providing a mixture comprising amphiphilic molecules and the hydrophobic linker; and

A: (a) contacting an aperture with the mixture, wherein a buffer comprising the second hydrophilic component is present on the trans side of the aperture, such that an amphiphilic layer comprising the hydrophobic linker forms across the aperture, and the second hydrophilic component attaches to the hydrophobic linker; and

(b) adding a buffer comprising the first hydrophilic component to the cis side of the amphiphilic layer such that the first hydrophilic component attaches to the hydrophobic linker;

or
B: (a) contacting an aperture with the mixture, wherein a buffer comprising the first hydrophilic component is present on the cis side of the aperture, such that an amphiphilic layer comprising the hydrophobic linker forms across the aperture, and the first hydrophilic component attaches to the hydrophobic linker; and

(b) adding a buffer comprising the second hydrophilic component to the trans side of the amphiphilic layer such that the second hydrophilic component attaches to the hydrophobic linker.

In some embodiments, the method comprises: (a) providing a mixture comprising amphiphilic molecules and the hydrophobic linker bound first to a first hydrophilic component; and (b) contacting an aperture with the mixture, wherein a buffer comprising a second hydrophilic component is present on the trans side of the aperture, such that an amphiphilic layer comprising the hydrophobic linker forms across the aperture, and the second hydrophilic component attaches to the hydrophobic linker on the trans side of the membrane.

In some embodiments, the method comprises (a) providing a mixture comprising amphiphilic molecules and the hydrophobic linker bound first to a second hydrophilic component; and (b) contacting an aperture with the mixture, wherein a buffer comprising a first hydrophilic component is present on the cis side of the aperture, such that an amphiphilic layer comprising the hydrophobic linker forms across the aperture, and the first hydrophilic component attaches to the hydrophobic linker on the cis side of the membrane.

Also provided herein is a method for assembling a tethering complex in an amphiphilic layer, wherein the tethering complex comprises a first hydrophilic component connected to a second hydrophilic component by a hydrophobic linker, the method comprising (i) forming the amphiphilic layer from a plurality of amphiphilic molecules; and (ii) contacting the amphiphilic layer with the hydrophobic linker; wherein the hydrophobic linker is optionally attached to either the first hydrophilic component or the second hydrophilic component.

In some embodiments the method further comprises attaching the first hydrophilic component and/or the second hydrophilic component to the hydrophobic linker once the amphiphilic layer has been formed thereby forming the tethering complex.

In some embodiments the method comprises contacting the first hydrophilic component; the second hydrophilic component; and the hydrophobic linker; wherein the first hydrophilic component comprises a first reactive group; the second hydrophilic component comprises a second reactive group; and the hydrophobic linker comprises reactive groups; and reacting the first reactive group with a reactive group on the hydrophobic linker and reacting the second reactive group with a reactive group on the hydrophobic linker so as to connect the first hydrophilic component to the second hydrophilic component by the hydrophobic linker, thereby forming the tethering complex.

Also provided is a method for assembling a tethering complex in an amphiphilic layer, wherein the tethering complex comprises a first hydrophilic component connected to a second hydrophilic component by a hydrophobic linker, the method comprising (a) contacting a first moiety with a second moiety, wherein the first moiety comprises the first hydrophilic component attached to a first hydrophobic moiety comprising a first reactive group and the second moiety comprises the second hydrophilic component attached to a second hydrophobic moiety comprising a second reactive group; and (b) reacting the first reactive group with the second reactive group thereby forming a hydrophobic linker connecting the first hydrophilic component to the second hydrophilic component, thereby forming the tethering complex.

In some embodiments the first hydrophilic component is provided from a first face of the amphiphilic layer and the second hydrophilic component is provided from a second face of the amphiphilic layer.

In some embodiments of the methods for assembling a tethering complex provided herein, the methods further comprise inserting a detector into the amphiphilic layer.

In some embodiments of the various methods described herein, the hydrophobic linker comprised in the tethering complex covalently attaches to (i) the one or more hydrophilic components or (ii) the first hydrophilic component and/or the second hydrophilic component. In some embodiments the hydrophobic linker non-covalently attaches to (i) the one or more hydrophilic components or (ii) the first hydrophilic component and/or the second hydrophilic component. In some embodiments (i) the hydrophobic linker covalently attaches to the first hydrophilic component and non-covalently attaches to the second hydrophilic component; or (ii) the hydrophobic linker non-covalently attaches to the first hydrophilic component and covalently attaches to the second hydrophilic component. In some embodiments the hydrophobic linker comprises or consists of a saturated or non-saturated hydrocarbon or organic molecule or a saturated or non-saturated inorganic molecule; wherein optionally the hydrophobic linker comprises or consists of a hydrophobic polypeptide, a spiroketal, polydimethylsiloxane (PDMS), an alkane, a protein, a transmembrane pore, a carbon nanotube, a natural lipid or a synthetic lipid-like molecule.

In some embodiments (i) at least one of the one or more hydrophilic components; or (ii) the first hydrophilic component comprises an analyte-binding moiety. In some embodiments (i) the analyte binding moiety comprises biotin and the first hydrophilic component comprises streptavidin; (ii) the analyte binding moiety comprises cholesterol and the first hydrophilic component comprises cyclodextrin; or (iii) the first hydrophilic component comprises a nucleotide or polynucleotide.

In some embodiments the second hydrophilic component comprises an anchor or anchor-binding moiety. In some embodiments (i) the anchor binding moiety comprises biotin and the anchor comprises streptavidin; or (ii) the anchor binding moiety comprises cholesterol and the anchor comprises cyclodextrin; or (iii) the anchor comprises a nucleotide or polynucleotide.

Also provided herein is a method of concentrating an analyte in the region of a detector, the method comprising:
concentrating a tethering complex in an amphiphilic layer as described herein; and
contacting the analyte with the tethering complex such that the analyte attaches to the first hydrophilic component of the tethering complex;
   thereby concentrating the analyte in the region of the detector.

In some embodiments, the analyte binds to a plurality of tethering complexes, thereby concentrating the analyte in the region of the detector.

Also provided herein is a method of concentrating an analyte in the region of a amphiphilic layer comprising a detector, the method comprising concentrating a plurality of tethering complexes in the region of the detector; and
i) contacting the analyte with said tethering complexes such that the analyte binds to a plurality of said tethering complexes; or
ii) contacting (A) a splint comprising (i) a plurality of binding sites for said tethering complexes and (ii) one or more binding sites for said analyte; and (B) the analyte with said tethering complexes such that the splint binds to a plurality of said tethering complexes and the analyte binds to the splint;
   thereby concentrating the analyte in the region of the detector.

In some embodiments, the tethering complexes and/or the amphiphilic layer are as defined herein.

Also provided herein is a method of characterising a target analyte; the method comprising concentrating the analyte in the region of a detector as described herein, and taking one or more measurements as the analyte moves with respect to the detector, wherein the one or more measurements are indicative of one or more characteristics of the analyte, and thereby characterising the analyte as it moves with respect to the detector.

In some embodiments, multiple target analytes are characterised. In some embodiments, the or each analyte is a polynucleotide, protein, peptide, carbohydrate or metabolite.

In those disclosed methods which relate to a detector, the detector in some embodiments comprises a transmembrane nanopore capable of characterising an analyte as the analyte moves with respect to the nanopore.

Also provided herein is an amphiphilic layer obtainable by a method described herein.

Also provided is an amphiphilic layer comprising a transmembrane nanopore and a tethering complex, wherein the tethering complex comprises a hydrophobic linker spanning the amphiphilic layer, a first hydrophilic component located on the cis side of the amphiphilic layer and a second hydrophilic component located on the trans side of the amphiphilic layer. In some embodiments the amphiphilic layer comprises a first region and a second region; wherein the first region differs chemically and/or physically from the second region, and wherein the nanopore is located in the first region and the tethering complex is concentrated in the first region.

In some embodiments, the provided amphiphilic layer is as defined in more detail here, and/or comprises a tethering complex as described in more detail here.

Also provided is an array comprising two or more amphiphilic layers as defined herein. Also provided is a device comprising the array, a means for applying a voltage potential across the amphiphilic layers and a means for detecting electrical charges across the amphiphilic layers. In some embodiments the device optionally further comprises a fluidics system for supplying a sample to the amphiphilic layer.

### BRIEF DESCRIPTION OF THE FIGURES

FIG. 1. Confocal micrograph showing the concentration of a fluorescently labelled polynucleotide by concentrating a tethering complex formed by non-covalent attachment in a first region of a membrane. A: No second hydrophilic component (“trans anchor”) is present in the tethering complex and the tethering complex does not localise in the first region of the amphiphilic layer. B: A second hydrophilic component (“trans anchor”) is present in the tethering complex and the tethering complex localises in the first region of the amphiphilic layer. Results are described in Example 1.

FIG. 2. Confocal micrograph showing the concentration of a fluorescently labelled polynucleotide by concentrating a tethering complex formed by covalent attachment in a first region of a membrane. A: No second hydrophilic component (“trans anchor”) is present in the tethering complex and the tethering complex does not localise in the first region of the amphiphilic layer. B: A second hydrophilic component (“trans anchor”) is present in the tethering complex and the tethering complex localises in the first region of the amphiphilic layer. C: A tethering complex having a shorter hydrophobic linker than in (B), and a DNA tether, is used. The tethering complex localises in the first region of the amphiphilic layer. Results are described in Example 2.

FIG. 3. Confocal micrograph showing the concentration of a fluorescently labelled polynucleotide by concentrating a tethering complex formed by covalent attachment in a first region of a membrane. A: No second hydrophilic component (“trans anchor”) is present in the tethering complex and the tethering complex does not localise in the first region of the amphiphilic layer. B: A second hydrophilic component (“trans anchor”) is present in the tethering complex and the tethering complex localises in the first region of the amphiphilic layer. Results are described in Example 3.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

It should be appreciated that “embodiments” of the disclosure can be specifically combined together unless the context indicates otherwise. The specific combinations of all disclosed embodiments (unless implied otherwise by the context) are further disclosed embodiments of the claimed invention.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a motor protein” includes two or more such proteins, reference to “a helicase” includes two or more helicases, reference to “a monomer” refers to two or more monomers, reference to “a pore” includes two or more pores and the like.

All publications, patents and patent applications cited herein, whether supra or infra, are hereby incorporated by reference in their entirety.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypeptides that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” has at least 50%, 60%, 70%, 80%, 90%, 95% or 99% complete sequence identity to the amino acid sequence of the corresponding wild-type protein. Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, charged (+) | Thr | polar, hydrophilic, neutral |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

### Disclosed Methods

The disclosure relates to methods of concentrating a tethering complex in a region of an amphiphilic layer.

The inventors have surprisingly found that tethering complexes which comprise one or more hydrophilic components connected by a hydrophobic linker can preferentially locate in a region of an amphiphilic layer comprising a nanopore, whereas known tethering complexes do not necessarily do so. As such, by using a tethering complex as described herein to anchor an analyte for detection by a nanopore to the membrane, the local concentration of the analyte in the vicinity of the nanopore can be improved.

Accordingly, provided herein is a method of concentrating a tethering complex in a region of an amphiphilic layer, said amphiphilic layer comprising a plurality of amphiphilic molecules and a detector, wherein the tethering complex comprises one or more components connected by a hydrophobic linker;

the method comprising contacting the tethering complex or one or more components thereof with said plurality of amphiphilic molecules;

and wherein the amphiphilic layer comprises a first region comprising the detector, and a second region, wherein the first region differs chemically and/or physically from the second region, and wherein the tethering complex preferentially localises to the first region relative to the second region; thereby concentrating the tethering complex in the first region of the amphiphilic layer.

Any suitable tethering complex comprising one or more hydrophilic components connected by a hydrophobic linker can be used in the disclosed methods. The tethering complex can be chosen or designed according to amphiphilic molecules in the amphiphilic layer. Alternatively, the amphiphilic molecules used to form the amphiphilic layer can be chosen or designed according to the tethering complex. Tethering complexes, and methods for their production, are described in more detail herein.

Any suitable amphiphilic molecules can be used to form the amphiphilic layer. Exemplary amphiphilic molecules are described in more detail herein.

In the disclosed method, the amphiphilic layer comprises a detector. Any suitable detector can be used in the disclosed methods. Suitable detectors include nanopores, as described in more detail herein.

In the disclosed methods, the amphiphilic layer comprises a first region and a second region. The first region comprises the detector. The second region does not comprise the detector. The first region differs chemically and/or physically from the second region. Any suitable methods can be used to partition the amphiphilic layer into first and second layers. Some suitable strategies are discussed in more detail herein.

Also disclosed herein is a method of concentrating an analyte in the region of a detector. Any suitable analyte that can be detected using a detector such as a nanopore can be investigated. Exemplary analytes are discussed in more detail herein.

Also disclosed is a method of characterising a target analyte. Any characteristics of the analyte that can be detected using a detector such as a nanopore can be determined using the disclosed methods. Suitable characteristics are discussed herein. Characterising the target analyte typically comprises taking one or more measurements characteristic of the analyte as the analyte moves with respect to the detector, e.g. the nanopore. The one or more measurements can be any suitable measurements. Typically, the one or more measurements are electrical measurements, e.g. current measurements, and/or are one or more optical measurements. Apparatuses for recording suitable measurements, and the information that such measurements can provide, are described in more detail herein.

### Concentrating the Tethering Complex

In developing the methods of the present disclosure, it has been found that when conventional coupling techniques are used to couple an analyte to a membrane, the concentration of the analyte in the vicinity of the nanopore is sometimes lower than might be expected or required based on the solution concentration. It was found that the tethers used to couple the analyte to the membrane typically do not localise solely in the region of the nanopore. This led to decreased efficiency in characterising analytes as their local concentration in the vicinity of the pore was reduced. The inventors sought to probe the origin of this effect, and in doing so developed the presently claimed methods.

Known devices for characterising analytes using nanopores typically comprise a chamber comprising an aqueous solution and a barrier that separates the chamber into two sections. The barrier typically has an aperture in which a membrane containing a transmembrane pore is formed.

To prepare such devices for characterising an analyte such as a polynucleotide, the aperture is typically coated with an oil such as hexadecane. The oil provides an interface between the solid substrate in which the aperture is formed, and the membrane which forms across the aperture. Once the aperture has been coated with the oil, amphiphilic molecules for forming a membrane (amphiphilic layer) may be applied.

The oil used to coat the aperture typically coats a significant portion of the apparatus apart from the aperture, for example the substrate in which the aperture is formed may also be coated with the oil. Without being bound by theory, the inventors consider that the origin of the reduced analyte concentration in the vicinity of the nanopore may be that the amphiphilic molecules used to form the amphiphilic layer do not locate only at the aperture, but rather typically coat all areas of the apparatus which are contacted by the oil.

It is known that amphiphilic membranes are typically naturally mobile, essentially acting as two dimensional fluids with lipid diffusion rates of approximately 10⁻⁸ cm s⁻¹. This means that unless otherwise constrained, components located with amphiphilic layer can move freely within the amphiphilic layer. Accordingly, and again without being bound by theory, the inventors considered that the tethers used in conventional methods to concentrate analyte molecules in the vicinity of the nanopore may therefore not concentrate in the vicinity of the nanopore, but instead may but diffuse over the entire available area accessible to the amphiphilic molecules, even though much of this area may be inaccessible to the nanopore. The inventors consider that this may cause analyte molecules to preferentially locate not only in the vicinity of the nanopore, but more widely across the area of the device which is accessible to the amphiphilic molecules, even if such areas are too remote from the nanopore for analytes located there to be characterised.

In developing the disclosed methods, it was found that this problem can be mitigated if the anchor used to localise the analyte in the region of the nanopore preferentially locates in the region of the nanopore. The disclosed methods thus provide the use tethering complexes which do so preferentially locate, and thus can be used to concentrate an analyte in a desired region, e.g. in the vicinity of a nanopore for its subsequent analysis, as well as such tethering complexes themselves.

The disclosed methods thus provide tethering complexes and their uses in localising an analyte in a desired region of an amphiphilic layer. The inventors have found that by using a tethering complex which comprises one or more hydrophilic components connected by a hydrophobic linker, the tethering complex can concentrate in the desired region. It has been found that such methods allow the analyte to similarly concentrate. The desired target region of the amphiphilic layer is a first region of the amphiphilic layer comprising a detector such as a nanopore. The tethering complex concentrates in the desired region relative to other non-desired “second” regions. The first region differs chemically and/or physically from the second region(s). As explained in more detail herein, the tethering complex can be designed to preferentially locate in first regions according to their properties including their amphiphilic molecule constituents.

In the disclosed methods, the tethering complex preferentially concentrates in an a first region of the amphiphilic layer, the first region also comprising a detector. The tethering complex does not preferentially concentrate in a second region of the amphiphilic layer.

In the disclosed methods, the tethering complex preferentially concentrates in the region of the amphiphilic layer comprising the detector, e.g. a nanopore. However, the tethering complex typically does not localise to the nanopore itself. In other words, the tethering complex localises in the amphiphilic molecules in the region of the detector, but typically is not bound to the detector. In some embodiments the tethering complex does not bind to the detector.

In the disclosed methods, the first region differs chemically and/or physically from the second region. In some embodiments the first region is a multi-layered region of the amphiphilic layer. In some embodiments, the first region and the second region both comprise the same type of amphiphilic molecules. In such embodiments, the properties of the first and second regions of the amphiphilic layer typically differ in physical form.

In some embodiments, the first region is a multi-layered region and the second region has a different number of layers to the first region. For example, in some embodiments the first region may consist of or comprise a bilayer, and the second region may consist of or comprise a monolayer. Thus, in some embodiments the amphiphilic first region comprises or consists of a bilayer of said amphiphilic molecules and the amphiphilic second region comprises or consists of a monolayer of said amphiphilic molecules.

In other embodiments, the first region and the second region may both consist of a bilayer, but the properties of the bilayer in the first and second region may differ. For example, in some embodiments additional components as well as amphiphilic molecules in the amphiphilic layer of the first region may cause the first region to have different properties to the amphiphilic layer of the second region. In other embodiments additional components as well as amphiphilic molecules in the amphiphilic layer of the second region may cause the second region to have different properties to the amphiphilic layer of the first region. The properties that may alter between the first and second regions of the amphiphilic layer may for example include its thickness. For example, the thickness of the amphiphilic layer in the second region may be greater than the thickness of the amphiphilic layer in the first region. Alternatively, the thickness of the amphiphilic layer in the first region may be greater than the thickness of the amphiphilic layer in the second region.

In some embodiments, the amphiphilic layer may be modified to form the first and second regions. In some embodiments, the amphiphilic layer may be modified to phase separate to form the first and second regions. Phase separation is the creation of two or more distinct phases from a single homogeneous mixture. For example, a 1:1 mixture of DOPC and sphingomyelin will phase-separate to form lipid rafts. Certain components preferentially locate in one or other of the phases. For example, the membrane protein PLAP (glycosylphosphatidylinositol-anchored protein placental alkaline phosphatase) preferentially inserts into raised sphingomyelin rafts compared to DOPC regions.

Thus, the first and second regions of the amphiphilic layer may be phase-separated regions of the amphiphilic layer. In some embodiments a partitioning agent may be used to induce phase separation in an amphiphilic layer. In some embodiments an agent which may modify the amphiphilic layer to form the first and second regions is selected from fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides. In some embodiments, examples of partitioning agents may include, for example, cholesterol and sphingomyelin.

In some embodiments, the first region and the second region may comprise different types of amphiphilic molecules. In other words, in some embodiments the first region comprises a first composition of amphiphilic molecules and the amphiphilic second region comprises a second composition of amphiphilic molecules, and the first composition differs from the second composition.

In some such embodiments, the first region may thus have different chemical properties to the first region. For example, the first region may comprise amphiphilic molecules which have hydrophilic headgroups and the second region may comprise amphiphilic molecules which have different hydrophilic headgroups. In this way the chemical reactivity of the first region may differ from that of the second region. For example, the hydrogen-bonding properties of the headgroups of the amphiphilic molecules in the first region may differ from that of the headgroups of the amphiphilic molecules in the second region.

In some embodiments the first region may have different physical properties to the first region. For example, the first region may comprise amphiphilic molecules with longer hydrophobic portions than the amphiphilic molecules in the second region. When the amphiphilic molecules are used to form an amphiphilic layer, a multi-layered amphiphilic layer may form. However the packing of the amphiphilic molecules in the first region and the second region will differ so that the amphiphilic molecules cluster in first and second regions. The amphiphilic layer (e.g. a bilayer) will then have different physical properties in the first region and the second region. For example, the thickness of the amphiphilic layer in the second region may be greater than the thickness of the amphiphilic layer in the first region. Alternatively, the thickness of the amphiphilic layer in the first region may be greater than the thickness of the amphiphilic layer in the second region.

In some embodiments the amphiphilic layer in the first region may differ both chemically and physically compared to the amphiphilic layer in the second region.

As explained in more detail below, in some embodiments the amphiphilic molecules in the amphiphilic layer are selected from lipids and copolymers.

Thus, in some embodiments the first region may consist of or comprise a lipid bilayer. In some embodiments the second region may consist of or comprise a lipid bilayer comprising different lipids to those in the first region. In some embodiments the second region may consist of or comprise a lipid monolayer. In some embodiments the lipids in the lipid bilayer of the first region are different from the lipids in the lipid bilayer or monolayer of the second region. In some embodiments the first region comprises or consists of a lipid bilayer and the second region comprises or consists of a lipid monolayer, and the lipids in the first region are the same as the lipids in the second region. In other words, in some embodiments, the amphiphilic multi-layered first region comprises a bilayer of said amphiphilic molecules and the amphiphilic second region comprises a monolayer of said amphiphilic molecules.

In some embodiments the first region may consist of or comprise a block copolymer layer. As explained in more detail herein, as used herein a block copolymer layer is a multi-layered structure. In some embodiments the second region may consist of or comprise a copolymer layer comprising different copolymers to those in the first region. In some embodiments the first region may comprise a bilayer of block copolymers and the second region may consist of or comprise a copolymer monolayer. In some embodiments the copolymers in the copolymer bilayer of the first region are different from the copolymers in the copolymer bilayer or monolayer of the second region. In some embodiments the first region comprises or consists of a copolymer layer and the second region comprises or consists of a copolymer layer, and the copolymers in the first region are the same as the copolymers in the second region.

In some embodiments the first region may consist of or comprise a lipid bilayer and the second region may consist of or comprise a block copolymer layer. In some embodiments the first region may consist of or comprise a block copolymer layer and the second region may consist of or comprise a lipid bilayer or monolayer.

In some embodiments the first region may comprise or consist of a lipid bilayer and the second region may comprise or consist of separated lipid monolayers. In some embodiments second region comprises or consists of lipid monolayers separated by a hydrophobic layer, e.g. an oil layer. In some embodiments the first region may comprise or consist of a block copolymer layer, and the second region may comprise or consist of separated block copolymer layers. In some embodiments the second region comprises or consists of lipid monolayers separated by a hydrophobic layer, e.g. an oil layer.

In some embodiments, the first region and the second region of the amphiphilic layer respectively correspond to first and second areas of a substrate, wherein first area of the substrate differs chemically and/or physically from the second area. For example, in some embodiments the first region may correspond to an aperture in a substrate.

In some embodiments the first region may correspond to a raised area on a substrate and the second region may correspond to a lowered area on a substrate. In some embodiments the first region may correspond to a lowered area on a substrate and the second region may correspond to a raised area on a substrate.

In some embodiments the first region may correspond to an area having different chemical properties to the second region. In some embodiments the first region is chemically treated to have different chemical properties to the second region. In some embodiments the second region is chemically treated to have different chemical properties to the first region. In some embodiments the first region and/or the second region are optionally coated portions of the substrate. In some embodiments the coating is an oil coating.

In some embodiments, the first region corresponds to the interfacial surface area between a first droplet and a second droplet pair, wherein the first and second droplets each have an amphiphilic coating; and the second region corresponds to the surface area of the portion of the first droplet which does not interface with a second droplet.

In some embodiments combinations of such features can be used. For example, in some embodiments (i) the first region may correspond to an aperture in a substrate and the second region corresponds to an optionally coated area of the substrate; and (ii) the first region comprises or consists of a lipid bilayer and the second region comprises or consists of a lipid monolayer or the first region comprises or consists of a block-copolymer layer and the second region comprises or consists of a block co-polymer layer which differs chemically and/or physically from the first region.

As will be apparent from the discussion herein, the methods comprise concentrating the tethering complex in the first region of the amphiphilic layer as compared to the second region. Any suitable means can be used to preferentially target the tethering complex to the first region of the amphiphilic layer.

In some embodiments the tethering complex is concentrated in the amphiphilic layer by controlling the physical properties of the tethering complex. In some embodiments the tethering complex is concentrated in the amphiphilic layer by controlling the physical properties of the tethering complex.

The tethering complex can be concentrated in the first region of the amphiphilic layer by controlling its physical properties. The tethering complex comprises one or more hydrophilic components linked by a hydrophobic linker. In some embodiments the tethering complex comprises a first hydrophilic component connected to a second hydrophilic component by a hydrophobic linker. One exemplary property that can be controlled is the length of the hydrophobic linker.

For example, in some embodiments the thickness of the amphiphilic layer in the first region differs from the thickness of the amphiphilic layer in the second region. By controlling the length of the hydrophobic linker in the tethering complex to correspond with the thickness of the amphiphilic layer in the first region, the tethering complex can preferentially localise in the first region.

For example, in some embodiments the first region of the amphiphilic layer is thinner than the amphiphilic layer in the second region. In such embodiments using a hydrophobic linker that can span the amphiphilic layer in the first region but not span the amphiphilic layer in the second region can cause the tethering complex to localise in the first region of the amphiphilic layer.

Without being bound by theory, it is believed that in some embodiments a hydrophobic linker that can span the amphiphilic layer in the first region but not span the amphiphilic layer in the second region can cause the tethering complex to localise in the first region of the amphiphilic layer due to the hydrophilic components attached to the hydrophobic linker. If the linker can span the amphiphilic layer then the hydrophilic components can extend beyond the typically hydrophobic core of the amphiphilic layer and extend into the typically aqueous medium. However, if the linker cannot span the amphiphilic layer then the energetic barrier to the hydrophilic components being present in the hydrophobic core of the amphiphilic layer is too great for the linker to be present in that region. Accordingly, the tethering complex localises in the first region.

In some embodiments the first region of the amphiphilic layer is thicker than the amphiphilic layer in the second region. In such embodiments using a hydrophobic linker that can span the amphiphilic layer in the first region will be longer than is necessary to span the amphiphilic layer in the second region. Again without being bound by theory it is considered that in some embodiments this can prevent the linker from packing well in the amphiphilic layer in the second region, and thus cause the tethering complex to localise in the first region of the amphiphilic layer.

Accordingly, in some embodiments, the tethering complex localises to the amphiphilic first region such that the hydrophobic linker spans the amphiphilic layer; and the one or more hydrophilic components extend from the amphiphilic layer. In some embodiments, the tethering complex comprises a first hydrophilic component connected to a second hydrophilic component by a hydrophobic linker, and the tethering complex localises to the amphiphilic first region such that the hydrophobic linker spans the amphiphilic layer; the first hydrophilic component extends from a first face of the amphiphilic layer and the second hydrophilic component extends from a second face of the amphiphilic layer.

In some embodiments the chemical properties of the hydrophobic linker can be controlled to localise the tethering complex in a first region of the amphiphilic layer. For example, in some embodiments the headgroups of the amphiphilic molecules in the first region of the amphiphilic layer can be chosen or determined to attract, attach to or react with the hydrophilic components in the tethering complex, whereas no such attraction, attachment or reaction occurs in the second region. Accordingly, it is preferential for the tethering complex to localise in the region of the first layer. In some embodiments the reaction is the formation of hydrogen bonds between the hydrophilic headgroups of the amphiphilic molecules in the first region of the amphiphilic layer and the hydrophilic components of the tethering complex.

In some embodiments, the tethering complex is concentrated in the first region of the amphiphilic layer by modifying the amphiphilic molecules in the second region of the amphiphilic layer to exclude the tethering complex from the second region.

In some embodiments, the tethering complex concentrates in the first region of the amphiphilic layer as the tethering complex or components thereof can diffuse from other regions of the amphiphilic layer (e.g. the second region of the amphiphilic layer) into the first region of the amphiphilic layer. For example, in some embodiments the hydrophobic linker diffuses into the first region of the amphiphilic layer. In some embodiments the hydrophobic linker attached to the one or more hydrophilic components diffuses into the first region of the amphiphilic layer. In some embodiments wherein the tethering complex comprises a first hydrophilic component connected to a second hydrophilic component by a hydrophobic linker, the hydrophobic linker attached to the first hydrophilic component diffuses into the first region of the amphiphilic layer. In some embodiments wherein the tethering complex comprises a first hydrophilic component connected to a second hydrophilic component by a hydrophobic linker, the hydrophobic linker attached to the second hydrophilic component diffuses into the first region of the amphiphilic layer. The diffusion of the hydrophobic linker optionally attached to a hydrophilic component can be localised in the first region of the amphiphilic layer by “capping” the non-bound end of the hydrophobic linker with a (further) hydrophilic component. For example, in some embodiments wherein the tethering complex comprises a first hydrophilic component connected to a second hydrophilic component by a hydrophobic linker, the diffusion of the hydrophobic linker optionally attached to the first hydrophilic component or the second hydrophilic component can be localised in the first region of the amphiphilic layer by “capping” the non-bound end of the hydrophobic linker with the second hydrophilic component or first hydrophilic component, as required.

Thus, in some embodiments the methods comprise allowing the hydrophobic linker to diffuse into the first region of the amphiphilic layer and then attaching one or more hydrophilic components to the hydrophobic linker.

In some embodiments wherein the tethering complex comprises a first hydrophilic component connected to a second hydrophilic component by a hydrophobic linker, the methods comprise allowing the hydrophobic linker to diffuse into the first region of the amphiphilic layer and then attaching the first hydrophilic component to the hydrophobic linker. In some embodiments the methods comprise allowing the hydrophobic linker attached to the second hydrophilic component to diffuse into the first region of the amphiphilic layer and then attaching the first hydrophilic component to the hydrophobic linker. In some embodiments the methods comprise allowing the hydrophobic linker to diffuse into the first region of the amphiphilic layer and then attaching the second hydrophilic component to the hydrophobic linker. In some embodiments the methods comprise allowing the hydrophobic linker attached to the first hydrophilic component to diffuse into the first region of the amphiphilic layer and then attaching the second hydrophilic component to the hydrophobic linker.

In some embodiments wherein the tethering complex comprises a first hydrophilic component connected to a second hydrophilic component by a hydrophobic linker:

(i) the first region may correspond to an aperture in a substrate and the second region corresponds to an optionally coated area of the substrate; e.g. an oil-coated area of the substrate;

(ii) the first region comprises or consists of a lipid bilayer and the second region comprises or consists of a lipid monolayer; or the first region comprises or consists of a block co-polymer layer and the second region comprises or consists of a block co-polymer layer, wherein the first region and second region differ chemically and/or physically;

(iii) the hydrophobic linker (optionally attached to at least one hydrophilic component) is allowed to diffuse from the second region of the amphiphilic layer into the first region of the amphiphilic layer; and

(iv) the hydrophobic linker is attached to a hydrophilic component to form the tethering complex and localise the tethering complex in the first region of the amphiphilic layer.

In some embodiments wherein the tethering complex comprises a first hydrophilic component connected to a second hydrophilic component by a hydrophobic linker:

(i) the first region may correspond to an aperture in a substrate and the second region corresponds to a optionally coated area of the substrate; e.g. an oil-coated area of the substrate;

(ii) the first region comprises or consists of a lipid bilayer and the second region comprises or consists of a lipid monolayer; or the first region comprises or consists of a block co-polymer layer and the second region comprises or consists of a block co-polymer layer, wherein the first region and second region differ chemically and/or physically;

(iii) the hydrophobic linker (optionally attached to the first hydrophilic component or the second hydrophilic component is allowed to diffuse from the second region of the amphiphilic layer into the first region of the amphiphilic layer); and

(iv) the hydrophobic linker is attached to the first hydrophilic component and the second hydrophilic component as required to form the tethering complex and localise the tethering complex in the first region of the amphiphilic layer.

In some embodiments the disclosed methods result in a significant increase in the concentration of the tethering complex in the first region of the nanopore as compared to the second region. For example, in some embodiments the concentration of the tethering complex in the first region is increased by a factor of from about 2 to about 10000, such as from about 10 to about 1000, e.g. from about 50 to about 500, e.g. about 100, relative to the concentration of the tethering complex in the second region.

### Linker

As explained above, the tethering complex comprises one or more hydrophilic components connected by a hydrophobic linker. In some embodiments, a first hydrophilic component is connected to the hydrophobic linker at a first end of the hydrophobic linker. In some embodiments a second hydrophilic component is connected to the hydrophobic linker at a second end of the hydrophobic linker. In some embodiments a first hydrophilic component is connected to the hydrophobic linker at a first end of the hydrophobic linker and a second hydrophilic component is connected to the hydrophobic linker at a second end of the hydrophobic linker

Any suitable linker can be used to connect the hydrophilic components. In some embodiments the hydrophobic linker is able to stably embed into a solvent in which the amphiphilic molecules are provided prior to formation of the amphiphilic layer. In some embodiments the hydrophobic linker is able to stably embed into an oil onto which the amphiphilic molecules are contacted prior to formation of the amphiphilic layer. In some embodiments the hydrophobic linker is soluble in the second region of the amphiphilic layer.

In some embodiments the hydrophobic linker is able to diffuse between the first and second regions of the amphiphilic layer. For example, in some embodiments the first region is a membrane region and the second region is an annulus around an aperture in a substrate. In some embodiments the annulus comprises an oil onto which the amphiphilic molecules are contacted prior to formation of the amphiphilic layer. In some embodiments the hydrophobic linker is able to diffuse between the annulus and the membrane region.

In some embodiments the hydrophobic linker is able to stably embed in the first region of the amphiphilic layer.

In some embodiments the hydrophobic linker has a length sufficient to span the first region of the amphiphilic layer.

In some embodiments the hydrophobic linker consists of or comprises a linear molecule or structure.

In some embodiments the hydrophobic linker comprises or consists of a saturated or non-saturated hydrocarbon or organic molecule or a saturated or non-saturated inorganic molecule.

In some embodiments the hydrophobic linker consists of or comprises a polymer. In some embodiments the polymer is selected from acrylics, amides and imides, carbonates, dienes, esters, ethers, fluorocarbons, olefins, styrenes, vinyl acetals, vinyl and vinylidene chlorides, vinyl esters, vinyl ethers and ketones, and vinylpyridine and vinypyrrolidone polymers. Hydrophobic polymers are commercially available from Sigma Aldrich (USA). In some embodiments the polymer may comprise from 2 to about 50 monomer acid units, such as from about 10 to about 30 monomers, e.g. about 20 monomers.

In some embodiments the hydrophobic linker consists of or comprises a hydrophobic polypeptide. A hydrophobic polypeptide can comprise natural or unnatural amino acids. Polypeptides are described in more detail herein. In some embodiments the hydrophobic polypeptide comprises or consists or hydrophobic amino acids. Hydrophobic amino acids include glycine (Gly), alanine (Ala), valine (Val), leucine (Leu), isoleucine (Ile), proline (Pro), phenylalanine (Phe), methionine (Met), and tryptophan (Trp). A hydrophobic polypeptide may comprise one or more hydrophilic peptide units providing the overall polypeptide is hydrophobic. In some embodiments the polypeptide may comprise from 2 to about 50 amino acid units, such as from about 10 to about 30 amino acids, e.g. about 20 amino acids.

In some embodiments the hydrophobic linker consists of or comprises a spiroketal.

In some embodiments the hydrophobic linker consists of or comprises a silicone. In some embodiments the silicone comprises a [SiR₂—O]n backbone wherein R is a hydrocarbyl group e.g. an alkane such as methyl. In some embodiments the silicone is polydimethylsiloxane (PDMS). In some embodiments the silicone is a PDMS of length from about 2 to about 200 monomer units.

In some embodiments the hydrophobic linker consists of or comprises a hydrocarbon. In some embodiments the hydrophobic linker consists of or comprises an alkane, alkene or alkyne. In some embodiments the alkane, alkene or alkyne has a length of from about 10 to about 100 carbon atoms, such as from about 25 to about 75 carbon atoms, e.g. from about 40 to about 60 atoms.

In some embodiments the hydrophobic linker consists of or comprises a protein. in some embodiments the hydrophobic linker consists of or comprises a transmembrane pore. In embodiments wherein the hydrophobic linker consists of or comprises a transmembrane pore, the transmembrane pore is not used as the detector. Thus, in embodiments wherein the hydrophobic linker consists of or comprises a first transmembrane pore, a second transmembrane pore is used as the detector. In such embodiments, the first transmembrane pore is typically different from the second transmembrane pore. Transmembrane pores are described in more detail herein.

In some embodiments the hydrophobic linker consists of or comprises a carbon nanotube. In some embodiments the carbon nanotube is a single-wall carbon nanotube. In some embodiments the carbon nanotube is a multi-walled carbon nanotube. Carbon nanotubes and their chemical modifications are well known in the art.

In some embodiments the hydrophobic linker consists of or comprises a natural lipid or a synthetic lipid-like molecule. Such molecules are described in more detail herein. In embodiments wherein the hydrophobic linker consists of or comprises a natural lipid or a synthetic lipid-like molecule, the natural lipid or a synthetic lipid-like molecule is not typically used as the amphiphilic molecules for producing the amphiphilic layer. Thus, in embodiments wherein the hydrophobic linker consists of or comprises a first natural lipid or a synthetic lipid-like molecule, second amphiphilic molecules are comprised in the amphiphilic layer. In such embodiments, the first lipid/lipid-type molecules are typically different to the amphiphilic molecules comprised in the amphiphilic layer. However in some embodiments the first lipid/lipid-type molecules are of the same type as the amphiphilic molecules comprised in the amphiphilic layer.

### First and Second Hydrophilic Components

In some embodiments at least one of the one or more hydrophilic components comprises or consists of an analyte-binding moiety. In some embodiments the analyte-binding moiety is a tether for the analyte. As such, the analyte can bind to the analyte-binding moiety and thus be concentrated where the tethering complex is concentrated in the methods disclosed herein.

Any suitable chemistry can be used to attach the analyte to the tether. In other words, the analyte-binding moiety can be any suitable binding moiety. Examples of suitable chemistry are discussed below. For example, in some embodiments the analyte binding moiety comprises biotin and a hydrophilic component (e.g. the first hydrophilic component) comprises streptavidin. In some embodiments the analyte binding moiety comprises streptavidin and a hydrophilic component (e.g. the first hydrophilic component) comprises biotin. In some embodiments the analyte binding moiety comprises cholesterol and a hydrophilic component (e.g. the first hydrophilic component) comprises a cyclodextrin. In some embodiments the analyte binding moiety comprises a cyclodextrin and a hydrophilic component (e.g. the first hydrophilic component) comprises cholesterol.

In some embodiments the analyte-binding moiety comprises or consists of a polynucleotide or polynucleotide. In some embodiments the analyte-binding moiety comprises or consists of a polynucleotide. In some embodiments the polynucleotide is complementary to the analyte or an adapter thereon so that the polynucleotide can attach to the analyte.

In some embodiments a second hydrophilic component comprises or consists of an anchor or anchor-binding moiety. In some embodiments the anchor or (an anchor bound to the anchor-binding moiety) does not cross the amphiphilic layer i.e. it cannot pass through the amphiphilic layer. In some embodiments the anchor (or an anchor bound to the anchor-binding moiety) does not perturb the amphiphilic layer.

Any suitable chemistry can be used to attach the anchor (or anchor-binding moiety) to the tether. In other words, the anchor-binding moiety can be any suitable binding moiety. Examples of suitable chemistry are discussed below.

For example, in some embodiments a second hydrophilic component comprises biotin, optionally linked to streptavidin. In some embodiments a second hydrophilic component comprises cholesterol, optionally linked to a cyclodextrin. Other examples of groups which form a strong (e.g. covalent) attachment which can be used to attach an anchor to the anchor-binding moiety include DBCO/azide, thiol/maleimide, thiol/dibromodiamide, transcyclooctene/tetrazine, and transcyclooctene/ortho-quinone.

In some embodiments a second hydrophilic component comprises or consists of a polynucleotide or polypeptide. In some embodiments a second hydrophilic component comprises or consists of a polynucleotide.

In some embodiments a second hydrophilic component comprises thiol, biotin or a surfactant. In some embodiments a second hydrophilic component comprises amylose (for binding to maltose binding protein or a fusion protein), Ni-NTA (for binding to poly-histidine or poly-histidine tagged proteins) or a peptide (such as an antigen).

In some embodiments a second hydrophilic component comprises a moiety that increases interaction of the second hydrophilic component with the amphiphilic layer. Suitable moieties can include, but are not limited to, hydrophobic groups that increase interaction with the amphiphilic layer, and affinity tags that interact with the amphiphilic molecules in the amphiphilic layer. In some embodiments the degree of interaction between the second hydrophilic component and the amphiphilic molecules and/or amphiphilic layer is used as a criteria for selecting or identifying an appropriate second hydrophilic component for use in the tethering complex.

In some embodiments wherein the tethering complex comprise a first hydrophilic component and a second hydrophilic component, the first hydrophilic component is different from the second hydrophilic component. In some embodiments it is advantageous that the hydrophobic linker of the tethering complex is attached to the first and second hydrophilic components using orthogonal chemistry. This can ensure that the linker attaches to exactly one first hydrophilic component and one second hydrophilic component (and does not inadvertently attach to two first hydrophilic components or two second hydrophilic components). In some embodiments it is advantageous that the hydrophobic linker of the tethering complex is attached to the first hydrophilic component from the cis side of the amphiphilic layer and to the second hydrophilic component from the trans side of the amphiphilic layer using orthogonal chemistry. This can ensure that the linker attaches to exactly one first hydrophilic component and one second hydrophilic component thereby ensuring the final tethering complex is formed in a transmembrane configuration.

Some non-limiting exemplary examples of orthogonal chemistries include:
(1) Biotin/streptavidin, and (2) cholesterol/cyclodextrin
(1) Biotin/streptavidin, and (2) DBCO/azide
(1) Thiol/maleimide, and (2) DBCO/azide.

### Assembling the Tethering Complex

The tethering complex used in the methods disclosed herein can be assembled in any suitable way. However, the disclosure also provides methods for assembling a tethering complex in an amphiphilic layer.

The disclosure provides a method for assembling a tethering complex in an amphiphilic layer, wherein the tethering complex comprises one or more hydrophilic components connected by a hydrophobic linker; the method comprising contacting the tethering complex or one or more components thereof with a plurality of amphiphilic molecules and subsequently forming the amphiphilic layer.

Also provided is a method for assembling a tethering complex in an amphiphilic layer, wherein the tethering complex comprises one or more hydrophilic components connected by a hydrophobic linker; the method comprising (i) forming the amphiphilic layer from a plurality of amphiphilic molecules; and (ii) contacting the amphiphilic layer with the tethering complex or one or more components thereof.

In these embodiments, the method may comprise (i) contacting the hydrophobic linker with the amphiphilic molecules or the amphiphilic layer; wherein the hydrophobic linker is not attached to at least one of the one or more hydrophilic components when the hydrophobic linker is contacted with the amphiphilic molecules or amphiphilic layer and (ii) attaching at least one of the one or more hydrophilic components to the hydrophobic linker once the amphiphilic layer has been formed, thereby forming the tethering complex.

In some embodiments the tethering complex can be assembled with at least one of the one or more hydrophilic components facing the cis face of the amphiphilic layer.

The disclosure also provides a method for assembling a tethering complex in an amphiphilic layer, wherein the tethering complex comprises a first hydrophilic component connected to a second hydrophilic component by a hydrophobic linker, the method comprising contacting the tethering complex or one or more components thereof with a plurality of amphiphilic molecules and subsequently forming the amphiphilic layer.

In some embodiments the tethering complex is assembled in the presence of the amphiphilic layer or components thereof. The tethering complex can be assembled so as to be oriented in the tethering complex with the first hydrophilic component facing a first face of the amphiphilic layer (e.g. the cis face) and the second hydrophilic component facing a second face of the amphiphilic layer (e.g. the trans face). Accordingly, in some embodiments the method of assembling the tethering complex comprises (i) contacting the hydrophobic linker with the plurality of amphiphilic molecules; and (ii) forming the amphiphilic layer.

In some embodiments the hydrophobic linker is attached to the first hydrophilic component prior to contacting the hydrophobic linker with the plurality of amphiphilic molecules. In some embodiments the hydrophobic linker is attached to the second hydrophilic component prior to contacting the hydrophobic linker with the plurality of amphiphilic molecules.

In some embodiments the hydrophobic linker is attached to both the first and second hydrophilic components prior to contacting the hydrophobic linker with the plurality of amphiphilic molecules. However, typically the hydrophobic linker is not attached to both the first and second hydrophilic components prior to contacting the hydrophobic linker with the plurality of amphiphilic molecules. Thus, in some of the method of assembling the tethering complex, the hydrophobic linker is not attached to the first hydrophilic component and/or the second hydrophilic component when the hydrophobic linker is contacted with the amphiphilic molecules, and the method further comprises attaching the first hydrophilic component and/or the second hydrophilic component to the hydrophobic linker once the amphiphilic layer has been formed thereby forming the tethering complex.

Accordingly, in some embodiments, (i) the hydrophobic linker is attached to one of the first and second hydrophilic components; (ii) the linker attached to one of the first and second hydrophilic components is mixed with the amphiphilic molecules; (iii) the amphiphilic layer is formed from the mixture; and (iv) the other of the first and second hydrophilic components is attached to the linker in the amphiphilic layer.

In some embodiments, assembling the tethering complex comprises providing a mixture comprising amphiphilic molecules and the hydrophobic linker; and

(a) contacting an aperture with the mixture, wherein a buffer comprising the second hydrophilic component is present on the trans side of the aperture, such that an amphiphilic layer comprising the hydrophobic linker forms across the aperture, and the second hydrophilic component attaches to the hydrophobic linker; and

(b) adding a buffer comprising the first hydrophilic component to the cis side of the amphiphilic layer such that the first hydrophilic component attaches to the hydrophobic linker.

In some embodiments, assembling the tethering complex comprises:

(a) providing a mixture comprising amphiphilic molecules and the hydrophobic linker bound first to a second hydrophilic component; and

(b) contacting an aperture with the mixture, wherein a buffer comprising a first hydrophilic component is present on the cis side of the aperture, such that an amphiphilic layer comprising the hydrophobic linker forms across the aperture, and the first hydrophilic component attaches to the hydrophobic linker on the cis side of the membrane.

In such embodiments, the hydrophobic linker, first hydrophilic component and second hydrophilic component are typically as described in more detail herein.

In some embodiments, assembling the tethering complex comprises providing a mixture comprising amphiphilic molecules and the hydrophobic linker; and

(a) contacting an aperture with the mixture, wherein a buffer comprising the first hydrophilic component is present on the cis side of the aperture, such that an amphiphilic layer comprising the hydrophobic linker forms across the aperture, and the first hydrophilic component attaches to the hydrophobic linker; and

(b) adding a buffer comprising the second hydrophilic component to the trans side of the amphiphilic layer such that the second hydrophilic component attaches to the hydrophobic linker.

In some embodiments, assembling the tethering complex comprises

(a) providing a mixture comprising amphiphilic molecules and the hydrophobic linker bound first to a first hydrophilic component; and

(b) contacting an aperture with the mixture, wherein a buffer comprising a second hydrophilic component is present on the trans side of the aperture, such that an amphiphilic layer comprising the hydrophobic linker forms across the aperture, and the second hydrophilic component attaches to the hydrophobic linker on the trans side of the membrane.

In such embodiments, the hydrophobic linker, first hydrophilic component and second hydrophilic component are typically as described in more detail herein.

Also provided is a method for assembling a tethering complex in an amphiphilic layer, wherein the tethering complex comprises a first hydrophilic component connected to a second hydrophilic component by a hydrophobic linker, the method comprising (i) forming the amphiphilic layer from a plurality of amphiphilic molecules; and (ii) contacting the amphiphilic layer with the hydrophobic linker.

In some embodiments the hydrophobic linker is attached to the first hydrophilic component. In some embodiments the hydrophobic linker is attached to the second hydrophilic component.

In some embodiments, the method further comprises attaching the first hydrophilic component to the hydrophobic linker once the amphiphilic layer has been formed thereby forming the tethering complex. In some embodiments, the method further comprises attaching the second hydrophilic component to the hydrophobic linker once the amphiphilic layer has been formed thereby forming the tethering complex. In some embodiments the method further comprises attaching the first hydrophilic component and the second hydrophilic component to the hydrophobic linker once the amphiphilic layer has been formed thereby forming the tethering complex.

In some embodiments, the method comprises contacting the first hydrophilic component; the second hydrophilic component; and the hydrophobic linker; wherein the first hydrophilic component comprises a first reactive group; the second hydrophilic component comprises a second reactive group; and the hydrophobic linker comprises reactive groups; and

reacting the first reactive group with a reactive group on the hydrophobic linker and reacting the second reactive group with a reactive group on the hydrophobic linker so as to connect the first hydrophilic component to the second hydrophilic component by the hydrophobic linker, thereby forming the tethering complex.

In some embodiments, provided herein is a method for assembling a tethering complex in an amphiphilic layer, wherein the tethering complex comprises a first hydrophilic component connected to a second hydrophilic component by a hydrophobic linker, the method comprising:

(a) contacting a first moiety with a second moiety, wherein the first moiety comprises the first hydrophilic component attached to a first hydrophobic moiety comprising a first reactive group and the second moiety comprises the second hydrophilic component attached to a second hydrophobic moiety comprising a second reactive group; and

(b) reacting the first reactive group with the second reactive group thereby forming a hydrophobic linker connecting the first hydrophilic component to the second hydrophilic component, thereby forming the tethering complex.

In some embodiments, the first hydrophilic component is provided from a first face of the amphiphilic layer and the second hydrophilic component is provided from a second face of the amphiphilic layer. For example, the first hydrophilic component may be provided from the cis face of the amphiphilic layer and the second hydrophilic component may be provided from the trans face of the amphiphilic layer. In some embodiments the reaction between the first moiety and the second moiety is a click chemistry reaction.

In some embodiments the method of assembling the tethering complex in an amphiphilic layer further comprises the step of inserting a detector such as a nanopore into the amphiphilic layer.

In the methods of assembling a tethering complex, any suitable buffer can be used. The buffer is typically in aqueous solution. Typically, the buffer is or comprises phosphate buffer. Other suitable buffers are HEPES and Tris-HCl buffer. Buffer components may be present at concentrations of from about 10 to about 50 mM, such as about 25 mM. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 8. Additional components may be present in the buffer solution such as ferrocyanide/ferricyanide anions and/or any aqueous soluble redox active couple(s).

In the methods of assembling a tethering complex which comprise reacting the hydrophobic linker with the first hydrophilic component and/or the second hydrophilic component, any suitable reactive groups can be used.

In some embodiments, the hydrophobic linker covalently attaches to the first hydrophilic component. In some embodiments the hydrophobic linker covalently attaches to the second hydrophilic component. In some embodiments the hydrophobic linker covalently attaches to the first hydrophilic component and the second hydrophilic component.

In some embodiments the hydrophobic linker has a reactive functional group which can be used to facilitate attachment to the first hydrophilic component and/or the second hydrophilic component. In some embodiments, the hydrophobic linker has a reactive functional group at its first end for covalent attachment to the first hydrophilic component. In some embodiments the hydrophobic linker has a reactive functional group at its second end for covalent attachment to the second hydrophilic component. In some embodiments the hydrophobic linker has a reactive functional group at its first end for covalent attachment to the first hydrophilic component and a reactive functional group at its second end for covalent attachment to the second hydrophilic component.

In some embodiments the hydrophobic linker non-covalently attaches to the first hydrophilic component. In some embodiments the hydrophobic linker non-covalently attaches to the second hydrophilic component. In some embodiments the hydrophobic linker non-covalently attaches to the first hydrophilic component and the second hydrophilic component.

In some embodiments, the hydrophobic linker has a ligand at its first end for non-covalent attachment to the first hydrophilic component. In some embodiments the hydrophobic linker has a ligand at its second end for non-covalent attachment to the second hydrophilic component. In some embodiments the hydrophobic linker has a ligand at its first end for non-covalent attachment to the first hydrophilic component and a ligand at its second end for non-covalent attachment to the second hydrophilic component.

In some embodiments, the hydrophobic linker covalently attaches to the first hydrophilic component and non-covalently attaches to the second hydrophilic component. In some embodiments, the hydrophobic linker non-covalently attaches to the first hydrophilic component and covalently attaches to the second hydrophilic component.

In some embodiments the hydrophobic linker has a reactive functional group at its first end for covalent attachment to the first hydrophilic component and a ligand at its second end for non-covalent attachment to the second hydrophilic component. In some embodiments the hydrophobic linker has a ligand at its first end for non-covalent attachment to the first hydrophilic component and a reactive functional group at its second end for covalent attachment to the second hydrophilic component.

Any suitable reactive groups and/or ligands can be used.

For example, a cysteine residue can be used to form a disulphide bond to the polynucleotide or to a modified group thereon. In some embodiments, the hydrophobic linker comprises a cysteine and the first hydrophilic component and/or the second hydrophilic component comprises a thiol, e.g. a cysteine.

In some embodiments the hydrophobic linker is modified in order to facilitate its attachment to the first hydrophilic component and/or the second hydrophilic component. For example, in some embodiments the hydrophobic linker is modified by attaching a moiety comprising a reactive functional group for attaching to the hydrophobic linker.

The attachment chemistry between the hydrophobic linker and the first hydrophilic component, and the hydrophobic linker and the second hydrophilic component, is not particularly limited. Any suitable combination of reactive functional groups can be used. In some embodiments the chemistry that attaches the hydrophobic linker to the first hydrophilic component is orthogonal to the chemistry that attaches the hydrophobic linker to the second hydrophilic component. As used herein, orthogonal chemistry relates to pairs of reactions which do not cross-react. In other words, in some embodiments the first end of the hydrophobic linker reacts with the first hydrophilic component but does not react with the second hydrophilic component, and the second end of the hydrophobic linker reacts with the second hydrophilic component but does not react with the first hydrophilic component. Examples of orthogonal chemistry include the reactions of thiols with maleimides and azides with alkynes. Such reactions can occur without any cross-reaction; i.e. thiols do not react with azides and maleimides do not react with alkynes.

Many suitable reactive groups and their chemical targets are known in the art. Some exemplary reactive groups and their corresponding targets include aryl azides which may react with amine, carbodiimides which may react with amines and carboxyl groups, hydrazides which may react with carbohydrates, hydroxymethyl phosphines which may react with amines, imidoesters which may react with amines, isocyanates which may react with hydroxyl groups, carbonyls which may react with hydrazines, maleimides which may react with sulfhydryl groups, NHS-esters which may react with amines, PFP-esters which may react with amines, psoralens which may react with thymine, pyridyl disulfides which may react with sulfhydryl groups, vinyl sulfones which may react with sulfhydryl amines and hydroxyl groups, and the like.

Another suitable chemistry for attaching the hydrophobic linker to the first hydrophilic component and/or the second hydrophilic component includes click chemistry Many suitable click chemistry reagents are known in the art. Suitable examples of click chemistry include, but are not limited to, the following:
(a) copper(I)-catalyzed azide-alkyne cycloadditions (azide alkyne Huisgen cycloadditions);
(b) strain-promoted azide-alkyne cycloadditions; including alkene and azide [3+2] cycloadditions; alkene and tetrazine inverse-demand Diels-Alder reactions; and alkene and tetrazole photoclick reactions;
(c) copper-free variant of the 1,3 dipolar cycloaddition reaction, where an azide reacts with an alkyne under strain, for example in a cyclooctane ring;
(d) the reaction of an oxygen nucleophile on one linker with an epoxide or aziridine reactive moiety on the other; and
(e) the Staudinger ligation, where the alkyne moiety can be replaced by an aryl phosphine, resulting in a specific reaction with the azide to give an amide bond.

Any reactive group may be used to form the conjugate. Some suitable reactive groups include [1, 4-Bis[3-(2-pyridyldithio)propionamido]butane; 1,1 1-bis-maleimidotriethyleneglycol; 3,3′-dithiodipropionic acid di(N-hydroxysuccinimide ester); ethylene glycol-bis(succinic acid N-hydroxysuccinimide ester); 4,4′-diisothiocyanatostilbene-2,2′-disulfonic acid disodium salt; Bis[2-(4-azidosalicylamido)ethyl] disulphide; 3-(2-pyridyldithio)propionic acid N-hydroxysuccinimide ester; 4-maleimidobutyric acid N-hydroxysuccinimide ester; lodoacetic acid N-hydroxysuccinimide ester; S-acetylthioglycolic acid N-hydroxysuccinimide ester; azide-PEG-maleimide; and alkyne-PEG-maleimide. The reactive group may be any of those disclosed in WO 2010/086602, particularly in Table 3 of that application.

In some embodiments the attachment between the hydrophobic linker and the first hydrophilic component and/or the second hydrophilic component is non-covalent. Examples of non-covalent attachment chemistry which can be used in the assembly of the tethering complex are given herein and include the non-covalent interaction of biotin with streptavidin; cyclodextrin with cholesterol, the interaction of a ligand with its receptor, the interaction of an antibody with an antigen, etc.

### Multivalent Binding

As explained above, the disclosed methods involve concentrating a tethering complex in a desired region of a nanopore.

In some embodiments it is desirable to maximise the strength of binding between an analyte of interest and the tethering complex. However, in some embodiments beneficial effects arise from controlling the strength of bonding. As explained below, weak bonding can be preferentially used in some embodiments in order to improve localisation of analytes.

The use of a tethering complex as described herein leads to the tethering complex preferentially localising in a desired region of the amphiphilic layer. However, the localisation or concentration of the tethering complex in the desired region of the amphiphilic layer may in some embodiments not be 100% efficient whilst still leading to effective concentration. In some embodiments, for example where localisation of the tethering complex in the desired region of the amphiphilic layer is not 100% efficient, it can be desirable to tune the strength of binding between the tethering complex and an analyte of interest

In some embodiments a strong binding is desired. This causes the analyte of interest to bind strongly to the tethering complex. This can be useful if the analyte is to repeatedly probed by the detector, for example, in a flossing mode.

However, in some embodiments a weak binding is desired. A weak binding means that the equilibrium of the analyte when bound to the tethering complex vs when not bound to the tethering complex shifts in favour of the non-bound state.

The present inventors have recognised that an analyte which only weakly binds to the tethering complex but which can bind to multiple tethering complexes can have significant advantages. This is particularly the case when relatively low concentrations of tethering complex are used, because the tethering complex preferentially localises in the desired region of the amphiphilic layer, as described herein.

For example, if an analyte which can strongly bind to the tethering complex encounters a tethering complex molecule which is not in the desired region of the amphiphilic layer, analyte may bind strongly to the tethering complex and thus be unavailable for sensing by the detector. However, if the binding of the analyte to the tethering complex is weak then the analyte will dissociate from the tethering complex and become available for binding to other tethering complex moieties which may be localised in the desired region of the amphiphilic layer. Because the tethering complex preferentially localises in the desired region of the amphiphilic layer, the concentration of tethering complex moieties in the desired region of the amphiphilic layer is greater than in the non-desired region and the analyte thus has a higher probability of encountering the tethering complex in the desired region of the amphiphilic layer. Accordingly, analyte will preferentially locate in the desired region of the amphiphilic layer. This is further enhanced when the analyte is capable of binding to multiple tethering complexes. Whilst each binding to each individual tethering complex may be weak, the cumulative binding strength to the plurality of tethering complexes which are bound by the analyte is greater and prevents the analyte from dissociating from the tethering complexes in the desired region of the amphiphilic layer. This strategy, referred to herein as “weak-and-multivalent attachment”, can lead to close to extremely high levels of localisation of the analyte in the desired region of the amphiphilic layer, and thus available for sensing by the detector. In the context of this approach, a “weak” binding typically relates to binding between a tethering complex and an analyte which is weaker than that which would be required if only monovalent attachment (i.e. one attachment point) was to be used.

Thus, in one embodiment, the tethering complex comprises an analyte-binding moiety and the analyte comprises a tethering complex-binding moiety. In some embodiments the strength of the binding between the analyte-binding moiety and the tethering complex-binding moiety on the analyte is relatively weak. In some embodiments the analyte comprises a plurality of tethering complex-binding moieties. In some embodiments the analyte is concentrated in a desired region of an amphiphilic layer, such as a region of an amphiphilic layer comprising a detector, by binding to multiple tethering complexes preferentially localised in the desired region of the amphiphilic layer.

In some embodiments the tethering complex comprises an analyte binding moiety which comprises an oligonucleotide. In some embodiments the or each tethering complex-binding moiety of the analyte comprises an oligonucleotide.

In some embodiments the oligonucleotide of the analyte-binding moiety of the tethering complex has a length of from about 2 to about 20 nucleotides, such as from about 5 to about 15 nucleotides, e.g. about 10 nucleotides. In some embodiments the oligonucleotide of the tethering complex-binding moiety of the analyte has a length of from about 2 to about 20 nucleotides, such as from about 5 to about 15 nucleotides, e.g. about 10 nucleotides. In some embodiments the oligonucleotide of the analyte-binding moiety of the tethering complex is complementary or substantially complementary to the oligonucleotide of the tethering complex-binding moiety of the analyte. Of course, other binding moieties can also be used in such aspects of the invention. Suitable binding pairs are disclosed herein.

In some embodiments, the strength of each binding in the multivalent binding corresponds to a melting temperature of about 10 to about 30° C., based on standard operating conditions for the methods provided herein of about 34° C.; more preferably the strength of each binding in the multivalent binding corresponds to a melting temperature of about 15 to about 30° C. such as from about 20 or about 25 to about 30° C. For example, two such binding sites could be used to bind the analyte or splint to two tethering complexes. By comparison, a strong single binding site could have a binding strength corresponding to a melting temperature of greater than about 35° C., such as at least 40° C., at least 45° C. or at least 50°, or more, under comparable conditions.

In some embodiments the analyte binds to a plurality of tethering complexes. In some embodiments the analyte binds to from 2 to 10 tethering complexes; such as from 2 to 5 tethering complexes, e.g. from 2 to 3 tethering complexes.

In some embodiments the analyte binds to the tethering complex or plurality of tethering complexes via a splint. A splint may for example comprise an oligonucleotide capable of binding to an polynucleotide analyte and capable of binding to a plurality of analyte-binding moieties on a plurality of tethering complexes. In some embodiments a splint may thus comprise an analyte binding moiety for binding to the analyte; and a plurality of tethering complex-binding moieties for binding to a plurality of tethering complexes. When the analyte-binding moiety of the tethering complex comprises an oligonucleotide such as an oligonucleotide described above, each tethering complex-binding moiety of the splint may comprise a complementary or substantially complementary polynucleotide sequence.

Accordingly, provided herein is a method of concentrating an analyte in the region of a amphiphilic layer comprising a detector, the method comprising concentrating a plurality of tethering complexes in the region of the detector; and
i) contacting the analyte with said tethering complexes such that the analyte binds to a plurality of said tethering complexes; or
ii) contacting (A) a splint comprising (i) a plurality of binding sites for said tethering complexes and (ii) one or more binding sites for said analyte; and (B) the analyte with said tethering complexes such that the splint binds to a plurality of said tethering complexes and the analyte binds to the splint;
   thereby concentrating the analyte in the region of the detector. In some embodiments the analyte, amphiphilic layer, detector, and/or tethering complexes are as further described herein.

In some embodiments each tethering complex comprises a first binding site for a splint. In some embodiments the splint comprises a plurality of second binding sites each capable of binding to a first binding site on a tethering complex; and a third binding site for binding an analyte. In some embodiments the analyte comprises a fourth binding site capable of binding to the third binding site.

In such embodiments, the analyte can be characterised by: (i) concentrating the tethering complex in the desired region of the amphiphilic layer, e.g in the region of a detector; (ii) contacting the splint with tethering complexes concentrated in the desired region of the amphiphilic layer thereby causing the splint to bind to a plurality of tethering complexes; and (iii) contacting the analyte with the splint thereby causing the analyte to bind to the splint. In some embodiments the splint is first contacted with the plurality of tethering complexes and the analyte is then contacted with the splint. In some embodiments the analyte is first contacted with the splint and the splint is then contacted with the plurality of tethering complexes.

Those skilled in the art will appreciate that a multiplicity of splints can be used in some embodiments. For example, a first splint may be used to bind to a plurality of tethering complexes concentrated in the desired region of an amphiphilic layer. A second splint may be used to bind to the first splint, and optionally one of more further splints may be used to bind to the second splint. The analyte may then bind to the second splint or to one or more of the further splints if present.

In some other embodiments each tethering complex comprises a first binding site for an analyte. In some embodiments the analyte comprises a plurality of second binding sites each capable of binding to the first binding site.

In such embodiments, the analyte can thus be characterised by: (i) concentrating the tethering complex in the desired region of the amphiphilic layer, e.g in the region of a detector; and (ii) contacting the analyte with tethering complexes concentrated in the desired region of the amphiphilic layer thereby causing the analyte to bind to a plurality of tethering complexes.

Those skilled in the art will appreciate that the weak-and-multivalent attachment strategy is applicable to tethering complexes as described herein, but also more widely to any anchor which preferentially locates in a desired region of an amphiphilic layer such as a membrane. Thus, also provided herein is a method of concentrating an analyte in a desired region of a membrane, said method comprising concentrating a membrane anchor in a desired region of a membrane; and directly or indirectly binding the analyte to a plurality of said membrane anchors, thereby concentrating said analyte in the desired region of a membrane. In some embodiments the desired region of a membrane comprises a detector as described herein, for example a nanopore as described herein. In some embodiments each membrane anchor comprises a tethering complex as described herein. In some embodiments said binding is direct binding. In some embodiments said binding is via a splint which binds to said plurality of membrane anchors and to the analyte. In some embodiments the binding of the analyte or splint to the membrane anchor or tethering complex is via hybridisation of oligonucleotide binding sites on the membrane anchor/tethering complex and the analyte or splint.

### Amphiphilic Layer

As explained above, the methods provided herein comprise concentrating a tethering complex in a region of an amphiphilic layer. The amphiphilic layer comprises a plurality of amphiphilic molecules and a detector such as a nanopore.

As used here, an amphiphilic layer, also referred to herein as a membrane, is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450).

In some embodiments the amphiphilic molecules comprised in the amphiphilic layer are copolymers. In some embodiments the amphiphilic layer is thus a block copolymer membrane.

Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this way, the block copolymer may possess amphiphilic properties and may form a structure (an amphiphilic layer) that mimics a biological membrane.

A block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. In some embodiments the amphiphilic layer is a triblock copolymer membrane.

As used herein, an amphiphilic layer formed from a diblock, triblock, tetrablock or pentablock copolymer is a “multi-layered” structure. The term “multi-layer” as used herein is used in its broadest sense to refer to a structure or system which comprises or consists of 2 or more constituent components which may or may not be homogenously arranged throughout the structure or system. For example, in some embodiments an amphiphilic layer formed from a triblock copolymer can be described as a three-layered structure. In some embodiments an amphiphilic layer formed from a tetrablock copolymer can be described as a four-layered structure.

Copolymer materials can be designed to mimic naturally occurring amphiphilic molecules. For example, archaebacterial bipolar tetraether lipids are naturally occurring lipids that are constructed such that the lipid forms a membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form an amphiphilic layer that behaves similarly to lipid bilayers and encompass a range of phase behaviours from vesicles through to laminar membranes. Amphiphilic layers formed from these triblock copolymers may hold advantages over biological lipid membranes. For example, because the triblock copolymer is synthesised, the exact construction can be carefully controlled to provide the correct chain lengths and properties required to form an amphiphilic layer having desired properties, e.g. that facilitates interaction with pores and other proteins.

Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes typically have increased mechanical and environmental stability compared with biological lipid membranes, for example a much higher operational temperature or pH range. The synthetic nature of the block copolymers provides a platform to customise polymer based membranes for a wide range of applications.

In some embodiments, the amphiphilic layer is one of the membranes disclosed in International Application No. WO2014/064443 or WO2014/064444, the entire contents of which are expressly incorporated in their entirety.

In some embodiments the amphiphilic layer is one of the membranes disclosed in U.S. Pat. No. 6,723,814, the entire contents of which are expressly incorporated in its entirety.

The amphiphilic layer may comprise lipid molecules. For example, the amphiphilic layer may comprise a lipid bilayer.

Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in WO 2008/102121, WO 2009/077734 and WO 2006/100484.

A lipid bilayer is formed from two opposing layers of lipids. As such, a lipid bilayer is a multi-layered structure, as used herein. The two layers of lipids are typically arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Methods for forming lipid bilayers are known in the art. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface. Liposomes can be formed by sonication, extrusion or the Mozafari method (Colas et al. (2007) Micron 38:841-847).

In some embodiments, a lipid bilayer may be formed as described in International Application No. WO 2009/077734. Advantageously in this method, the lipid bilayer is formed from dried lipids. In a most preferred embodiment, the lipid bilayer is formed across an opening as described in WO2009/077734.

In the disclosed methods, any lipid that forms an amphiphilic layer e.g. a lipid bilayer may be used as the amphiphilic molecules. The amphiphilic molecules to be used in the amphiphilic layer can be chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

Lipid molecules lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The amphiphilic molecules used in the disclosed methods may be chemically-modified or functionalised. Both copolymers and lipids can be chemically-modified.

The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalised PEG Lipids, such as 1,2-Distearoyl-sn-Glycero-3 Phosphoethanolamine-N-[Biotinyl(Polyethylene Glycol)2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine.

The amphiphilic molecules used in the disclosed methods may be chemically-modified or functionalised in any suitable manner. For example, they may be chemically modified to facilitate interaction with the tethering complex. They may be chemically modified to couple to an analyte, e.g. an analyte attached to the tethering complex.

In some embodiments, the amphiphilic layer, for example the lipid composition, may comprise one or more additives that affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

An amphiphilic layer may be formed, for example, in or across a solid state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. A solid state layer may be formed from graphene. Suitable graphene layers are disclosed in WO 2009/035647. If a solid state layer is used, the detector e.g. a nanopore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The methods disclosed herein are typically carried out using (i) an artificial amphiphilic layer comprising a pore, (ii) an isolated, naturally-occurring amphiphilic layer (e.g. formed of naturally-occurring lipids) comprising a pore, (iii) a cell having a pore inserted therein; or (iv) an artificial amphiphilic layer, such as an artificial triblock copolymer layer. The layer may comprise other transmembrane and/or intramembrane proteins as well as other molecules in addition to the pore.

### Detector

As explained above, the methods disclosed herein comprise concentrating a tethering complex in a region of an amphiphilic layer comprising a detector. The disclosure also provides methods of concentrating an analyte in the region of a detector, and methods of characterising a target analyte using a detector.

In the disclosed methods, any suitable detector can be used. The detector may be selected from (i) a zero-mode waveguide, (ii) a field-effect transistor, optionally a nanowire field-effect transistor; (iii) an AFM tip; (iv) a nanotube, optionally a carbon nanotube; and (v) a nanopore. Preferably, the detector is a nanopore.

Aspects of the disclosure relate to detecting an analyte. An analyte may be characterised in the methods provided herein in any suitable manner. In one embodiment the analyte is characterised by detecting an ionic current or optical signal as the analyte moves with respect to a nanopore. This is described in more detail herein. The method is amenable to these and other methods of detecting analytes.

In another non-limiting example, in one embodiment the analyte is a polynucleotide and is characterised by detecting the by-products of a polynucleotide-processing reaction, such as a sequencing by synthesis reaction. The method may thus involve detecting the product of the sequential addition of (poly)nucleotides by an enzyme such as a polymerase to the nucleic acid strand. The product may be a change in one or more properties of the enzyme such as in the conformation of the enzyme. Such methods may thus comprise subjecting an enzyme such as polymerase or a reverse transcriptase to a double-stranded polynucleotide under conditions such that the template-dependent incorporation of nucleotide bases into a growing oligonucleotide strand causes conformational changes in the enzyme in response to sequentially encountering template strand nucleic acid bases and/or incorporating template-specified natural or analog bases (i.e., an incorporation event), detecting the conformational changes in the enzyme in response to such incorporation events, and thereby detecting the sequence of the template strand. In such methods the polynucleotide strand may be moved in accordance with the methods provided herein. Such methods may involve detecting and/or measuring incorporation events using methods known to those skilled in the art, such as those described in US 2017/0044605.

In another embodiment, by-products may be labelled so that a phosphate labelled species is released upon the addition of a nucleotide to a synthesised nucleic acid strand that is complementary to the template strand, and the phosphate labelled species is detected e.g. using a detector as described herein. The polynucleotide being characterised in this way may be moved in accordance with the methods herein. Suitable labels may be optical labels that are detected using a nanopore, or a zero mode wave guide, or by Raman spectroscopy, or other detectors. Suitable labels may be non-optical labels that are detected using a nanopore, or other detectors.

In another approach, nucleoside phosphates (nucleotides) are not labelled and upon the addition of a nucleotide to a synthesised nucleic acid strand that is complementary to the template strand, a natural by-product species is detected. Suitable detectors may be ion-sensitive field-effect transistors, or other detectors.

These and other detection methods are suitable for use in the methods described herein.

### Nanopore

In some embodiments the detector is a nanopore. In one embodiment a nanopore is a transmembrane pore.

A transmembrane pore is a structure that crosses a membrane to some degree. It permits hydrated ions driven by an applied potential to flow across or within the membrane. A transmembrane pore typically crosses the entire membrane so that hydrated ions may flow from one side of the membrane to the other side of the membrane. However, a transmembrane pore does not have to cross the membrane. It may be closed at one end. For instance, the pore may be a well, gap, channel, trench or slit in the membrane along which or into which hydrated ions may flow.

Any transmembrane pore may be used in the methods provided herein. The pore may be biological or artificial. Suitable pores include, but are not limited to, protein pores, polynucleotide pores and solid state pores. The pore may be a DNA origami pore (Langecker et al., Science, 2012; 338: 932-936). Suitable DNA origami pores are disclosed in WO2013/083983.

In one embodiment, the detector is a transmembrane protein pore. A transmembrane protein pore is a polypeptide or a collection of polypeptides that permits hydrated ions, such as polynucleotide, to flow from one side of a membrane to the other side of the membrane. In the methods provided herein, the transmembrane protein pore is capable of forming a pore that permits hydrated ions driven by an applied potential to flow from one side of the membrane to the other. The transmembrane protein pore preferably permits polynucleotides to flow from one side of the membrane, such as a triblock copolymer membrane, to the other. The transmembrane protein pore allows a polynucleotide to be moved through the pore.

In one embodiment, the detector is a transmembrane protein pore which is a monomer or an oligomer. The pore is preferably made up of several repeating subunits, such as at least 6, at least 7, at least 8, at least 9, at least 10, at least 11, at least 12, at least 13, at least 14, at least 15, or at least 16 subunits. The pore is preferably a hexameric, heptameric, octameric or nonameric pore. The pore may be a homo-oligomer or a hetero-oligomer.

In one embodiment, a transmembrane protein pore comprises a barrel or channel through which the ions may flow. The subunits of the pore typically surround a central axis and contribute strands to a transmembrane β-barrel or channel or a transmembrane α-helix bundle or channel.

Typically, the barrel or channel of a transmembrane protein pore comprises amino acids that facilitate interaction with an analyte, such as a target polynucleotide (as described herein). These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

In one embodiment, a nanopore is a transmembrane protein pore derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. Suitable α-helix bundle pores include, but are not limited to, inner membrane proteins and a outer membrane proteins, such as WZA and ClyA toxin.

In one embodiment a nanopore is a transmembrane pore derived from or based on Msp, α-hemolysin (α-HL), lysenin, CsgG, ClyA, Sp1 or haemolytic protein fragaceatoxin C (FraC).

In one embodiment, a nanopore is a transmembrane protein pore derived from CsgG, e.g. from CsgG from E. coli Str. K-12 substr. MC4100. Such a pore is oligomeric and typically comprises 7, 8, 9 or 10 monomers derived from CsgG. The pore may be a homo-oligomeric pore derived from CsgG comprising identical monomers. Alternatively, the pore may be a hetero-oligomeric pore derived from CsgG comprising at least one monomer that differs from the others. Examples of suitable pores derived from CsgG are disclosed in WO 2016/034591.

In one embodiment, a nanopore is a transmembrane pore derived from lysenin. Examples of suitable pores derived from lysenin are disclosed in WO 2013/153359.

In one embodiment, a nanopore is a transmembrane pore derived from or based on α-hemolysin (α-HL). The wild type α-hemolysin pore is formed of 7 identical monomers or sub-units (i.e., it is heptameric). An α-hemolysin pore may be α-hemolysin-NN or a variant thereof. The variant preferably comprises N residues at positions E111 and K147.

In one embodiment a nanopore is a transmembrane protein pore derived from leukocidin. A leukocidin is a hetero-oligomeric pore with two different subunits, one class S subunit and one class F subunit. Suitable leukocidins include, but are not limited to, gamma hemolysin (g-HL) comprising LukF (HlgB) and Hlg2 (HlgA), leukocidin comprising LukF (HlgB) and LukS(HlgC), leukocidin PV comprising LukF-PV and LukS-PV, LukE/LukD pore comprising LukE and LukD and LukS-I/LukF-I comprising LukF-I and LukS-I.

In one embodiment, a nanopore is a transmembrane protein pore derived from Msp, e.g. from MspA. Examples of suitable pores derived from MspA are disclosed in WO 2012/107778.

In one embodiment, a nanopore is a transmembrane pore derived from or based on ClyA.

### Analyte

The analyte can be any suitable substance for characterizing in the methods disclosed herein. Suitable analytes include, but are not limited to, metal ions, inorganic salts, polymers, such as a polymeric acids or bases, dyes, bleaches, pharmaceuticals, diagnostic agents, recreational drugs, explosives and environmental pollutants. In some embodiments the or each analyte is a polynucleotide, protein, peptide, carbohydrate or metabolite.

In some embodiments the analyte is modified for attachment to a tethering complex. In some embodiments the analyte is selected for its affinity to a tethering complex. In some embodiments the analyte has an adapter attached thereto for attachment to the tethering complex.

In some embodiments the analyte is a polynucleotide. Any suitable polynucleotide can be characterised in the disclosed methods.

In some embodiments the polynucleotide is secreted from cells. Alternatively, the polynucleotide can be produced inside cells such that it must be extracted from cells for characterisation in the disclosed methods.

A polynucleotide may be provided as an impure mixture of one or more polynucleotides and one or more impurities. Impurities may comprise truncated forms of polynucleotides which are distinct from the target polynucleotide for characterisation in the disclosed methods. For example the polynucleotide for characterisation in the disclosed methods may be genomic DNA and impurities may comprise fractions of genomic DNA, plasmids, etc. The target polynucleotide may be a coding region of genomic DNA and undesired polynucleotides may comprise non-coding regions of DNA.

Examples of polynucleotides include DNA and RNA. The bases in DNA and RNA may be distinguished by their physical size.

A polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas.

One or more nucleotides in the polynucleotide may be modified, for instance with a label or a tag, for which suitable examples are known by a skilled person. The polynucleotide may comprise one or more spacers. An adapter, for example a sequencing adapter, may be comprised in the polynucleotide. Adapters, tags and spacers are described in more detail herein.

Examples of modified bases are disclosed herein and can be incorporated into the polynucleotide by means known in the art, e.g. by polymerase incorporation of modified nucleotide triphosphates during strand copying (e.g. in PCR) or by polymerase fill-in methods. In some embodiments one or more bases can be modified by chemical means using reagents known in the art.

A nucleotide typically contains a nucleobase, a sugar and at least one phosphate group. The nucleobase and sugar form a nucleoside. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC). The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. The nucleotides in the polynucleotide may be attached to each other in any manner. The nucleotides are typically attached by their sugar and phosphate groups as in nucleic acids. The nucleotides may be connected via their nucleobases as in pyrimidine dimers.

A polynucleotide may be double stranded or single stranded.

In some embodiments the polynucleotide is single stranded DNA. In some embodiments the polynucleotide is single stranded RNA. In some embodiments the polynucleotide is a single-stranded DNA-RNA hybrid. DNA-RNA hybrids can be prepared by ligating single stranded DNA to RNA or vice versa. The polynucleotide is most typically single stranded deoxyribonucleic acid (DNA) or single stranded ribonucleic nucleic acid (RNA).

In some embodiments the polynucleotide is double stranded DNA. In some embodiments the polynucleotide is double stranded RNA. In some embodiments the polynucleotide is a double-stranded DNA-RNA hybrid. Double-stranded DNA-RNA hybrids can be prepared from single-stranded RNA by reverse transcribing the cDNA complement.

The polynucleotide can be any length. For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length.

Nucleotides can have any identity, and include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP. A nucleotide may be abasic (i.e. lack a nucleobase). A nucleotide may also lack a nucleobase and a sugar (i.e. is a C3 spacer).

The polynucleotide may comprise the products of a PCR reaction, genomic DNA, the products of an endonuclease digestion and/or a DNA library. The polynucleotide may be obtained from or extracted from any organism or microorganism. The polynucleotide may be obtained from a human or animal, e.g. from urine, lymph, saliva, mucus, seminal fluid or amniotic fluid, or from whole blood, plasma or serum. The polynucleotide may be obtained from a plant e.g. a cereal, legume, fruit or vegetable. The polynucleotide may comprise genomic DNA. The genomic DNA may be fragmented. The DNA may be fragmented by any suitable method. For example, methods of fragmenting DNA are known in the art, Such methods may use a transposase, such as a MuA transposase. Often the genomic DNA is not fragmented.

In some embodiments the analyte is a polypeptide. Any suitable polypeptide can be characterised in the disclosed methods.

In some embodiments the polypeptide is an unmodified protein or a portion thereof, or a naturally occurring polypeptide or a portion thereof.

In some embodiments the polypeptide is secreted from cells. Alternatively, the polypeptide can be produced inside cells such that it must be extracted from cells for characterisation by the disclosed methods. The polypeptide may comprise the products of cellular expression of a plasmid, e.g. a plasmid used in cloning of proteins in accordance with the methods described in Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016).

The polypeptide may be obtained from or extracted from any organism or microorganism. The polypeptide may be obtained from a human or animal, e.g. from urine, lymph, saliva, mucus, seminal fluid or amniotic fluid, or from whole blood, plasma or serum. The polypeptide may be obtained from a plant e.g. a cereal, legume, fruit or vegetable.

The polypeptide can be provided as an impure mixture of one or more polypeptides and one or more impurities. Impurities may comprise truncated forms of the target polypeptide which are distinct from the “target polypeptides” for characterisation in the disclosed methods. For example, the target polypeptide may be a full length protein and impurities may comprise fractions of the protein. Impurities may also comprise proteins other than the target protein e.g. which may be co-purified from a cell culture or obtained from a sample.

A polypeptide may comprise any combination of any amino acids, amino acid analogs and modified amino acids (i.e. amino acid derivatives). Amino acids (and derivatives, analogs etc) in the polypeptide can be distinguished by their physical size and charge.

The amino acids/derivatives/analogs can be naturally occurring or artificial.

In some embodiments the polypeptide may comprise any naturally occurring amino acid. Twenty amino acids are encoded by the universal genetic code. These are alanine (A), arginine (R), asparagine (N), aspartic acid (D), cysteine (C), glutamic acid/glutamate (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y) and valine (V). Other naturally occurring amino acids include selenocysteine and pyrrolysine.

In some embodiments the polypeptide is modified. In some embodiments the polypeptide is modified for detection using the disclosed methods. In some embodiments the disclosed methods are for characterising modifications in the target polypeptide.

In some embodiments one or more of the amino acids/derivatives/analogs in the polypeptide is modified. In some embodiments one or more of the amino acids/derivatives/analogs in the polypeptide is post-translationally modified. As such, the methods disclosed herein can be used to detect the presence, absence, number of positions of post-translational modifications in a polypeptide. The disclosed methods can be used to characterise the extent to which a polypeptide has been post-translationally modified.

Any one or more post-translational modifications may be present in the polypeptide. Typical post-translational modifications include modification with a hydrophobic group, modification with a cofactor, addition of a chemical group, glycation (the non-enzymatic attachment of a sugar), biotinylation and pegylation. Post-translational modifications can also be non-natural, such that they are chemical modifications done in the laboratory for biotechnological or biomedical purposes. This can allow monitoring the levels of the laboratory made peptide, polypeptide or protein in contrast to the natural counterparts.

Examples of post-translational modification with a hydrophobic group include myristoylation, attachment of myristate, a C₁₄ saturated acid; palmitoylation, attachment of palmitate, a C₁₆ saturated acid; isoprenylation or prenylation, the attachment of an isoprenoid group; farnesylation, the attachment of a farnesol group; geranylgeranylation, the attachment of a geranylgeraniol group; and glypiation, and glycosylphosphatidylinositol (GPI) anchor formation via an amide bond.

Examples of post-translational modification with a cofactor include lipoylation, attachment of a lipoate (C₈) functional group; flavination, attachment of a flavin moiety (e.g. flavin mononucleotide (FMN) or flavin adenine dinucleotide (FAD)); attachment of heme C, for instance via a thioether bond with cysteine; phosphopantetheinylation, the attachment of a 4′-phosphopantetheinyl group; and retinylidene Schiff base formation.

Examples of post-translational modification by addition of a chemical group include acylation, e.g. O-acylation (esters), N-acylation (amides) or S-acylation (thioesters); acetylation, the attachment of an acetyl group for instance to the N-terminus or to lysine; formylation; alkylation, the addition of an alkyl group, such as methyl or ethyl; methylation, the addition of a methyl group for instance to lysine or arginine; amidation; butyrylation; gamma-carboxylation; glycosylation, the enzymatic attachment of a glycosyl group for instance to arginine, asparagine, cysteine, hydroxylysine, serine, threonine, tyrosine or tryptophan; polysialylation, the attachment of polysialic acid; malonylation; hydroxylation; iodination; bromination; citrulination; nucleotide addition, the attachment of any nucleotide such as any of those discussed above, ADP ribosylation; oxidation; phosphorylation, the attachment of a phosphate group for instance to serine, threonine or tyrosine (O-linked) or histidine (N-linked); adenylylation, the attachment of an adenylyl moiety for instance to tyrosine (O-linked) or to histidine or lysine (N-linked); propionylation; pyroglutamate formation; S-glutathionylation; Sumoylation; S-nitrosylation; succinylation, the attachment of a succinyl group for instance to lysine; selenoylation, the incorporation of selenium; and ubiquitinilation, the addition of ubiquitin subunits (N-linked).

In some embodiments the polypeptide contains one or more cross-linked sections, e.g. C—C bridges. In some embodiments the polypeptides is not cross-linked prior to being characterised using the disclosed methods.

In some embodiments the polypeptide comprises sulphide-containing amino acids and thus has the potential to form disulphide bonds. Typically, in such embodiments, the polypeptide is reduced using a reagent such as DTT (Dithiothreitol) or TCEP (tris(2-carboxyethyl)phosphine) prior to being characterised using the disclosed methods.

The polypeptide can be a polypeptide of any suitable length. In some embodiments the polypeptide has a length of at least 10, at least 50, at least 100, at least 150, at least 200, at least 250, at least 300, at least 400, at least 500 or at least 1000 peptide units. In some embodiments the polypeptide has a length of from about 2 to about 1000 peptide units, for example from about 10 to about 500 peptide units, e.g. from about 30 to about 300 peptide units, such as from about 50 to about 200 peptide units, e.g. from about 100 to about 150 peptide units.

The analyte may be a polysaccharide produced by a bacterium such as a pathogenic bacterium. The polysaccharide may be a capsular polysaccharide having a molecular weight of 100-2000 kDa. The polysaccharide may be synthesized from nucleotide-activated precursors (called nucleotide sugars). The polysaccharide may be a lipopolysaccharide. The polysaccharide may be a therapeutic polysaccharide. The polysaccharide may be a toxic polysaccharide. The polysaccharide may be suitable for use as a vaccine. The polysaccharide may be for example bacterial or derived from a plant. The polysaccharide may be useful as an antibiotic, such as streptomycin, neomycins, paromomycine, kanamycin, chalcomycin, erythromycin, magnamycin, spiramycin, oleandomycin, cinerubin and amicetin, or a derivative of any one of the preceding compounds. The polysaccharide may be a sugar. The polysaccharide may be a polysaccharide such as callose, amylose, laminarin, chrysolaminarin, xylan, arabinoxylan, mannan, galactomannan, dextran, levan, inulin, polygalactosamine, gellan, xanthan, cellulose, glucomannan, helicellulose, chitin, chitosan, hyaluronic acid, elsinan, pullulan, etc.

Any number of analytes can be characterised in the disclosed methods. In some embodiments multiple target analytes are characterised. For instance, the method may comprise characterising 2, 3, 4, 5, 6, 7, 8, 9, 10, 20, 30, 50, 100 or more analytes. If two or more analytes are characterised, they may be different analytes or two instances of the same analytes. The analytes can be naturally occurring or artificial.

It is within the scope of the methods provided herein that the analyte is labelled with a molecular label. A molecular label may be a modification to the analyte which promotes the detection of the analyte in the methods provided herein. For example the label may be a modification to the analyte which alters the signal obtained as the analyte is characterised. For example, the label may interfere with a flux of ions through the nanopore. In such a manner, the label may improve the sensitivity of the methods.

Some embodiments disclosed herein comprise characterising a target analyte. In some embodiments such methods comprise concentrating the analyte in the region of a detector using the methods disclosed herein. In some embodiments one or more measurements are taken as the analyte moves with respect to the detector, wherein the one or more measurements are indicative of one or more characteristics of the analyte, and thereby characterising the analyte as it moves with respect to the detector.

It will be apparent from the above discussion that the measurements taken in the disclosed methods are typically characteristic of one or more characteristics of the analyte. For example, the identity of the analyte can be determined. In some embodiments the analyte is a polynucleotide. In some such embodiments the characteristics detected in the disclosed methods are selected from (i) the length of the polynucleotide, (ii) the identity of the polynucleotide, (iii) the sequence of the polynucleotide, (iv) the secondary structure of the polynucleotide and (v) whether or not the polynucleotide is modified. In some embodiments the analyte is a polypeptide. In some such embodiments the characteristics detected in the disclosed methods are selected from (i) the length of the polypeptide, (ii) the identity of the polypeptide, (iii) the sequence of the polypeptide, (iv) the secondary structure of the polypeptide and (v) whether or not the polypeptide is modified.

In typical embodiments the measurements are characteristic of the sequence of the polynucleotide or polypeptide.

### Adapters

In embodiments of the methods provided herein in which the analyte is a polynucleotide, the polynucleotide may have a polynucleotide adapter attached thereto. An adapter typically comprises a polynucleotide strand capable of being attached to the end of the polynucleotide.

In some embodiments the adapter is attached to the polynucleotide before the polynucleotide is attached to the tethering complex. In some embodiments the adapter is attached to the polynucleotide after the polynucleotide is attached to the tethering complex.

Accordingly, in some embodiments the methods comprise attaching an adapter (e.g. an adapter as described herein) to the polynucleotide and attaching the polynucleotide to the tethering complex. In some embodiments the methods comprise attaching the polynucleotide to the tethering complex and attaching an adapter (e.g. an adapter as described herein) to the polynucleotide thus attached to the tethering complex.

In some embodiments the adapter may be chosen or modified in order to provide a specific site for the conjugation to the polynucleotide.

An adapter may be attached to just one end of the polynucleotide. A polynucleotide adapter may be added to both ends of the polynucleotide. Alternatively, different adapters may be added to the two ends of the polynucleotide.

Adapters may be added to both strands of double stranded polynucleotides. Adapter may be added to single stranded polynucleotides. Methods of adding adapters to polynucleotides are known in the art. Adapters may be attached to polynucleotides, for example, by ligation, by click chemistry, by tagmentation, by topoisomerisation or by any other suitable method.

In one embodiment, the or each adapter is synthetic or artificial. Typically, the or each adapter comprises a polymer as described herein. In some embodiments, the or each adapter comprises a spacer as described herein. In some embodiments, the or each adapter comprises a polynucleotide. The or each polynucleotide adapter may comprise DNA, RNA, modified DNA (such as abasic DNA), RNA, PNA, LNA, BNA and/or PEG. Usually, the or each adapter comprises single stranded and/or double stranded DNA or RNA. The adapter may comprise the same type of polynucleotide as the polynucleotide strand to which it is attached. The adapter may comprise a different type of polynucleotide to the polynucleotide strand to which it is attached. In some embodiments the polynucleotide strand used in the disclosed methods is a single stranded DNA strand and the adapter comprises DNA or RNA, typically single stranded DNA. In some embodiments the polynucleotide is a double stranded DNA strand and the adapter comprises DNA or RNA, e.g. double or single stranded DNA.

In some embodiments, an adapter may be a bridging moiety. A bridging moiety may be used to connect the two strands of a double-stranded polynucleotide. For example, in some embodiments a bridging moiety is used to connect the template strand of a double stranded polynucleotide to the complement strand of the double stranded polynucleotide.

A bridging moiety typically covalently links the two strands of a double-stranded polynucleotide. The bridging moiety can be anything that is capable of linking the two strands of a double-stranded polynucleotide, provided that the bridging moiety does not interfere with movement of the polynucleotide with respect to the nanopore. Suitable bridging moieties include, but are not limited to a polymeric linker, a chemical linker, a polynucleotide or a polypeptide. Preferably, the bridging moiety comprises DNA, RNA, modified DNA (such as abasic DNA), RNA, PNA, LNA or PEG. The bridging moiety is more preferably DNA or RNA.

In some embodiments a bridging moiety is a hairpin adapter. A hairpin adapter is an adapter comprising a single polynucleotide strand, wherein the ends of the polynucleotide strand are capable of hybridising to each other, or are hybridized to each other, and wherein the middle section of the polynucleotide forms a loop. Suitable hairpin adapters can be designed using methods known in the art. In some embodiments a hairpin loop is typically 4 to 100 nucleotides in length, e.g. from 4 to 50 such as from 4 to 20 e.g. from 4 to 8 nucleotides in length. In some embodiments the bridging moiety (e.g. hairpin adapter) is attached at one end of a double-stranded polynucleotide. A bridging moiety (e.g. hairpin adapter) is typically not attached at both ends of a double-stranded polynucleotide.

In some embodiments, an adapter is a linear adapter. A linear adapter may be bound to either or both ends of a single stranded polynucleotide. When the polynucleotide is a double stranded polynucleotide, a linear adapter may be bound to either or both ends of either or both strands of the double stranded polynucleotide. A linear adapter may comprise a leader sequence as described herein. A linear adapter may comprise a portion for hybridisation with a tag (such as a pore tag) as described herein. A linear adapter may be 10 to 150 nucleotides in length, such as from 20 to 120, e.g. 30 to 100, for example 40 to 80 such as 50 to 70 nucleotides in length. A linear adapter may be single stranded. A linear adapter may be double stranded.

In some embodiments, an adapter may be a Y adapter. A Y adapter is typically a polynucleotide adapter. A Y adapter is typically double stranded and comprises (a) at one end, a region where the two strands are hybridised together and (b), at the other end, a region where the two strands are not complementary. The non-complementary parts of the strands typically form overhangs. The presence of a non-complementary region in the Y adapter gives the adapter its Y shape since the two strands typically do not hybridise to each other unlike the double stranded portion. The two single-stranded portions of the Y adapter may be the same length, or may be different lengths. For example, one single-stranded portion of the Y adapter may be 10 to 150 nucleotides in length, such as from 20 to 120, e.g. 30 to 100, for example 40 to 80 such as 50 to 70 nucleotides in length and the other single stranded portion of the Y adapter may independently by 10 to 150 nucleotides in length, such as from 20 to 120, e.g. 30 to 100, for example 40 to 80 such as 50 to 70 nucleotides in length. The double-stranded “stem” portion of the Y adapter may be e.g. from 10 to 150 nucleotides in length, such as from 20 to 120, e.g. 30 to 100, for example 40 to 80 such as 50 to 70 nucleotides in length.

An adapter may be linked to the polynucleotide by any suitable means known in the art. The adapter may be synthesized separately and chemically attached or enzymatically ligated to the polynucleotide. Alternatively, the adapter may be generated in the processing of the polynucleotide. In some embodiments, the adapter is linked to the polynucleotide at or near one end of the polynucleotide. In some embodiments, the adapter is linked to the polynucleotide within 50, e.g. within 20 for example within 10 nucleotides of an end of the polynucleotide. In some embodiments the adapter is linked to the polynucleotide at a terminus of the polynucleotide. When a adapter is linked to the polynucleotide the adapter may comprise the same type of nucleotides as the polynucleotide or may comprise different nucleotides to the polynucleotide.

### Spacers

In some embodiments of the methods provided herein in which the analyte is a polynucleotide, the polynucleotide or an adapter as described herein, may comprise a spacer. For example, one or more spacers may be present in the polynucleotide adapter. For example, the polynucleotide adapter may comprise from one to about 20 spacers, e.g. from about 1 to about 10, e.g. from 1 to about 5 spacers, e.g. 1, 2, 3, 4 or 5 spacers. The spacer may comprise any suitable number of spacer units. A spacer may provide an energy barrier which impedes movement of a polynucleotide-handling protein. For example, a spacer may stall a polynucleotide-handling protein by reducing the traction of the polynucleotide-handling protein on the polynucleotide. This may be achieved for instance by using an abasic spacer i.e. a spacer in which the bases are removed from one or more nucleotides in the polynucleotide adapter. A spacer may physically block movement of a polynucleotide-handling protein, for instance by introducing a bulky chemical group to physically impede the movement of the polynucleotide-handling protein.

In some embodiments, one or more spacers are included in a polynucleotide analyte or in an adapter as used in the methods claimed herein in order to provide a distinctive signal when they pass through or across the nanopore, i.e. as they move with respect to the nanopore.

In some embodiments, a spacer may comprise a linear molecule, such as a polymer. Typically, such a spacer has a different structure from the polynucleotide used in the conjugate. For instance, if the polynucleotide analyte is DNA, the or each spacer typically does not comprise DNA. In particular, if the polynucleotide analyte is deoxyribonucleic acid (DNA) or ribonucleic acid (RNA), the or each spacer preferably comprises peptide nucleic acid (PNA), glycerol nucleic acid (GNA), threose nucleic acid (TNA), locked nucleic acid (LNA) or a synthetic polymer with nucleotide side chains. In some embodiments, a spacer may comprise one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more C₃ (OC₃H₆OPO₃) groups, one or more photo-cleavable (PC) [OC₃H₆-C(O)NHCH₂-C₆H₃NO₂—CH(CH₃)OPO₃] groups, one or more hexandiol groups, one or more spacer 9 (iSp9) [(OCH₂CH₂)₃OPO₃] groups, or one or more spacer 18 (iSp18) [(OCH₂CH₂)₆OPO₃] groups; or one or more thiol connections. A spacer may comprise any combination of these groups. Many of these groups are commercially available from IDT® (Integrated DNA Technologies®). For example, C3, iSp9 and iSp18 spacers are all available from IDT®. A spacer may comprise any number of the above groups as spacer units.

In some embodiments, a spacer may comprise one or more chemical groups which cause a polynucleotide-handling protein to stall. In some embodiments, suitable chemical groups are one or more pendant chemical groups. The one or more chemical groups may be attached to one or more nucleobases in the polynucleotide analyte or adapter. The one or more chemical groups may be attached to the backbone of the polynucleotide adapter. Any number of appropriate chemical groups may be present, such as 2, 3, 4, 5, 6, 7, 8, 9, 10, 11, 12 or more. Suitable groups include, but are not limited to, fluorophores, streptavidin and/or biotin, cholesterol, methylene blue, dinitrophenols (DNPs), digoxigenin and/or anti-digoxigenin and dibenzylcyclooctyne groups. In some embodiments, a spacer may comprise a polymer. In some embodiments the spacer may comprise a polymer which is a polypeptide or a polyethylene glycol (PEG).

In some embodiments, a spacer may comprise one or more abasic nucleotides (i.e. nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic spacers can be inserted into target polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). In one embodiment, the one or more spacers do not comprise any abasic nucleotides.

### Tags

In some embodiments of the methods provided herein in which the detector is a nanopore (discussed above), a tag on the nanopore can be used, e.g. to promote the capture of an analyte by the nanopore.

The interaction between a tag on a nanopore and the binding site on an analyte such as a polynucleotide (e.g., the binding site present in a polynucleotide or in an adaptor attached to the polynucleotide, wherein the binding site can be provided by an anchor or a leader sequence of an adaptor or by a capture sequence within the duplex stem of an adaptor) may be reversible. For example, a polynucleotide can bind to a tag on a nanopore, e.g., via its adaptor, and release at some point, e.g., during characterization of the polynucleotide by the nanopore and/or during processing by a motor protein. A strong non-covalent bond (e.g., biotin/avidin) is still reversible and can be useful in some embodiments of the methods described herein. For example, a pair of pore tag and polynucleotide adaptor can be designed to provide a sufficient interaction between the complement of a double stranded polynucleotide (or a portion of an adaptor that is attached to the complement) and the nanopore such that the complement is held close to the nanopore (without detaching from the nanopore and diffusing away) but is able to release from the nanopore as it is processed.

A pore tag and polynucleotide adaptor can be configured such that the binding strength or affinity of a binding site on the polynucleotide (e.g., a binding site provided by an anchor or a leader sequence of an adaptor or by a capture sequence within the duplex stem of an adaptor) to a tag on a nanopore is sufficient to maintain the coupling between the nanopore and polynucleotide until an applied force is placed on it to release the bound polynucleotide from the nanopore.

In some embodiments, a pore tag is uncharged. This can ensure that it is not drawn into the nanopore under the influence of a potential difference if present.

One or more molecules that attract or bind the analyte or adaptor may be linked to the detector, e.g. to the nanopore. Any molecule that hybridizes to the analyte or adaptor may be used. The molecule may be selected from a PNA tag, a PEG linker, a short oligonucleotide, a positively charged amino acid and an aptamer. Nanopores having such molecules linked to them are known in the art. For example, pores having short oligonucleotides attached thereto are disclosed in Howarka et al (2001) Nature Biotech. 19: 636-639 and WO 2010/086620, and pores comprising PEG attached within the lumen of the pore are disclosed in Howarka et al (2000) J. Am. Chem. Soc. 122(11): 2411-2416.

A short oligonucleotide attached to a nanopore, which comprises a sequence complementary to a sequence in the conjugate (e.g. in a leader sequence or another single stranded sequence in an adaptor) may be used to enhance capture of the analyte in the methods described herein.

In some embodiments, a pore tag may comprise or be an oligonucleotide (e.g., DNA, RNA, LNA, BNA, PNA, or morpholino). The oligonucleotide can have about 10-30 nucleotides in length or about 10-20 nucleotides in length. In some embodiments, the oligonucleotide can have at least one end (e.g., 3′- or 5′-end) modified for conjugation to other modifications or to a solid substrate surface including, e.g., a bead. The end modifiers may add a reactive functional group which can be used for conjugation. Examples of functional groups that can be added include, but are not limited to amino, carboxyl, thiol, maleimide, aminooxy, and any combinations thereof. The functional groups can be combined with different length of spacers (e.g., C3, C9, C12, Spacer 9 and 18) to add physical distance of the functional group from the end of the oligonucleotide sequence.

Examples of modifications on the 3′ and/or 5′ end of oligonucleotides include, but are not limited to 3′ affinity tag and functional groups for chemical linkage (including, e.g., 3′-biotin, 3′-primary amine, 3′-disulfide amide, 3′-pyridyl dithio, and any combinations thereof); 5′ end modifications (including, e.g., 5′-primary ammine, and/or 5′-dabcyl), modifications for click chemistry (including, e.g., 3′-azide, 3′-alkyne, 5′-azide, 5′-alkyne), and any combinations thereof.

In some embodiments, a pore tag may further comprise a polymeric linker, e.g., to facilitate coupling to the nanopore. An exemplary polymeric linker includes, but is not limited to polyethylene glycol (PEG). The polymeric linker may have a molecular weight of about 500 Da to about 10 kDa (inclusive), or about 1 kDa to about 5 kDa (inclusive). The polymeric linker (e.g., PEG) can be functionalized with different functional groups including, e.g., but not limited to maleimide, NHS ester, dibenzocyclooctyne (DBCO), azide, biotin, amine, alkyne, aldehyde, and any combinations thereof.

Other examples of pore tags include, but are not limited to, His tags, biotin or streptavidin, antibodies that bind to analytes, aptamers that bind to analytes, analyte binding domains such as DNA binding domains (including, e.g., peptide zippers such as leucine zippers, single-stranded DNA binding proteins (SSB)), and any combinations thereof.

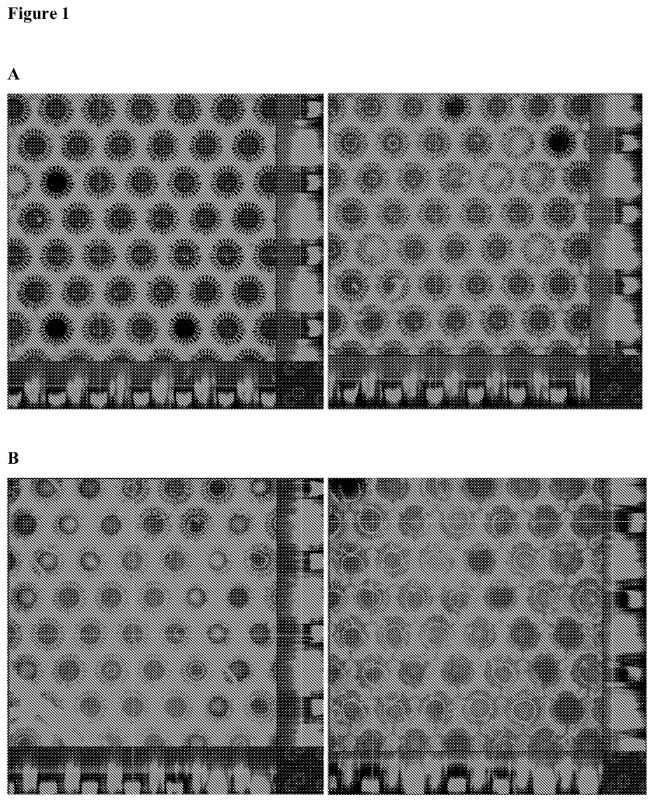

A pore tag may be attached to the external surface of a nanopore, e.g., on the cis side of a membrane, using any methods known in the art. For example, one or more tags can be attached to a nanopore via one or more cysteines (cysteine linkage), one or more primary amines such as lysines, one or more non-natural amino acids, one or more histidines (His tags), one or more biotin or streptavidin, one or more antibody-based tags, one or more enzyme modification of an epitope (including, e.g., acetyl transferase), and any combinations thereof. Suitable methods for carrying out such modifications are well-known in the art. Suitable non-natural amino acids include, but are not limited to, 4-azido-L-phenylalanine (Faz) and any one of the amino acids numbered 1-71 in FIG. 1 of Liu C. C. and Schultz P. G., Annu. Rev. Biochem., 2010, 79, 413-444.

In some embodiments where one or more tags are attached to a nanopore via cysteine linkage(s), the one or more cysteines can be introduced to one or more monomers that form the nanopore by substitution. In some embodiments, a nanopore may be chemically modified by attachment of (i) Maleimides including diabromomaleimides such as: 4-phenylazomaleinanil, 1.N-(2-Hydroxyethyl)maleimide, N-Cyclohexylmaleimide, 1.3-Maleimidopropionic Acid, 1.1-4-Aminophenyl-1H-pyrrole,2,5,dione, 1.1-4-Hydroxyphenyl-1H-pyrrole,2,5,dione, N-Ethylmaleimide, N-Methoxycarbonylmaleimide, N-tert-Butylmaleimide, N-(2-Aminoethyl)maleimide, 3-Maleimido-PROXYL, N-(4-Chlorophenyl)maleimide, 1-[4-(dimethylamino)-3,5-dinitrophenyl]-1H-pyrrole-2,5-dione, N-[4-(2-Benzimidazolyl)phenyl]maleimide, N-[4-(2-benzoxazolyl)phenyl]maleimide, N-(1-naphthyl)-maleimide, N-(2,4-xylyl)maleimide, N-(2,4-difluorophenyl)maleimide, N-(3-chloro-para-tolyl)-maleimide, 1-(2-amino-ethyl)-pyrrole-2,5-dione hydrochloride, 1-cyclopentyl-3-methyl-2,5-dihydro-1H-pyrrole-2,5-dione, 1-(3-aminopropyl)-2,5-dihydro-1H-pyrrole-2,5-dione hydrochloride, 3-methyl-1-[2-oxo-2-(piperazin-1-yl)ethyl]-2,5-dihydro-1H-pyrrole-2,5-dione hydrochloride, 1-benzyl-2,5-dihydro-1H-pyrrole-2,5-dione, 3-methyl-1-(3,3,3-trifluropropyl)-2,5-dihydro-1H-pyrrole-2,5-dione, 1-[4-(methylamino)cyclohexyl]-2,5-dihydro-1H-pyrrole-2,5-dione trifluroacetic acid, SMILES O═C1C═CC(═O)N1CC=2C═CN═CC2, SMILES O═C1C═CC(═O)N1CN2CCNCC2, 1-benzyl-3-methyl-2,5-dihydro-1H-pyrrole-2,5-dione, 1-(2-fluorophenyl)-3-methyl-2,5-dihydro 1H-pyrrole-2,5-dione, N-(4-phenoxyphenyl)maleimide, N-(4-nitrophenyl)maleimide (ii) lodocetamides such as: 3-(2-Iodoacetamido)-proxyl, N-(cyclopropylmethyl)-2-iodoacetamide, 2-iodo-N-(2-phenylethyl)acetamide, 2-iodo-N-(2,2,2-trifluoroethyl)acetamide, N-(4-acetylphenyl)-2-iodoacetamide, N-(4-(aminosulfonyl)phenyl)-2-iodoacetamide, N-(1,3-benzothiazol-2-yl)-2-iodoacetamide, N-(2,6-diethylphenyl)-2-iodoacetamide, N-(2-benzoyl-4-chlorophenyl)-2-iodoacetamide, (iii) Bromoacetamides: such as N-(4-(acetylamino)phenyl)-2-bromoacetamide, N-(2-acetylphenyl)-2-bromoacetamide, 2-bromo-n-(2-cyanophenyl)acetamide, 2-bromo-N-(3-(trifluoromethyl)phenyl)acetamide, N-(2-benzoylphenyl)-2-bromoacetamide, 2-bromo-N-(4-fluorophenyl)-3-methylbutanamide, N-Benzyl-2-bromo-N-phenylpropionamide, N-(2-bromo-butyryl)-4-chloro-benzenesulfonamide, 2-Bromo-N-methyl-N-phenylacetamide, 2-bromo-N-phenethyl-acetamide,2-adamantan-1-yl-2-bromo-N-cyclohexyl-acetamide, 2-bromo-N-(2-methylphenyl)butanamide, Monobromoacetanilide, (iv) Disulphides such as: aldrithiol-2, aldrithiol-4, isopropyl disulfide, 1-(Isobutyldisulfanyl)-2-methylpropane, Dibenzyl disulfide, 4-aminophenyl disulfide, 3-(2-Pyridyldithio)propionic acid, 3-(2-Pyridyldithio)propionic acid hydrazide, 3-(2-Pyridyldithio)propionic acid N-succinimidyl ester, am6amPDP1-βCD and (v) Thiols such as: 4-Phenylthiazole-2-thiol, Purpald, 5,6,7,8-tetrahydro-quinazoline-2-thiol.

In some embodiments, a pore tag may be attached directly to a nanopore or via one or more linkers. The tag may be attached to the nanopore using the hybridization linkers described in WO 2010/086602. Alternatively, peptide linkers may be used. Peptide linkers are amino acid sequences. The length, flexibility and hydrophilicity of the peptide linker are typically designed such that it does not to disturb the functions of the monomer and pore. A peptide linker may be a stretch of 2 to 20, such as 4, 6, 8, 10 or 16, serine and/or glycine amino acids. In some embodiments a flexible linker includes (SG)i, (SG)2, (SG)3, (SG)4, (SG)5 and (SG)8 wherein S is serine and G is glycine. In some embodiments a rigid linker is a stretch of 2 to 30, such as 4, 6, 8, 12, 16 or 24, prolines.

### Controlling Movement of the Analyte

As explained in more detail above, some embodiments of the disclosed methods comprise characterising an analyte. The analyte can be characterised as it moves with respect to a detector, e.g. a nanopore.

The movement of the analyte with respect to the detector may be driven by any suitable means. In some embodiments, the movement of the analyte is driven by a physical or chemical force (potential). In some embodiments the physical force is provided by an electrical (e.g. voltage) potential or a temperature gradient, etc.

In some embodiments, the analyte moves with respect to the detector (e.g. with respect to a nanopore) as an electrical potential is applied across the detector (e.g. across the nanopore). Analytes such as polynucleotides are negatively charged, and so applying a voltage potential across a nanopore will cause the analyte to move with respect to the nanopore under the influence of the applied voltage potential. For example, if a positive voltage potential is applied to the trans side of the nanopore relative to the cis side of the nanopore, then this will induce a negatively charged analyte to move from the cis side of the nanopore to the trans side of the nanopore. Similarly, if a positive voltage potential is applied to the trans side of the nanopore relative to the cis side of the nanopore then this will impede the movement of a negatively charged analyte from the trans side of the nanopore to the cis side of the nanopore. The opposite will occur if a negative voltage potential is applied to the trans side of the nanopore relative to the cis side of the nanopore. Apparatuses and methods of applying appropriate voltages are described in more detail herein.

In some embodiments the chemical force is provided by a concentration (e.g. pH) gradient.

In some embodiments, the movement of the analyte is driven by an analyte-handling enzyme. For example, in embodiments wherein the analyte is a biological polymer such as a polynucleotide or polypeptide, the movement of the polymer may be controlled by polynucleotide-handling or polypeptide-handling enzyme.

In some embodiments wherein the analyte is a polynucleotide, a polynucleotide-handling protein controls the movement of the analyte with respect to the detector.

Suitable polynucleotide-handling proteins are also known as motor proteins or polynucleotide-handling enzymes. Suitable polynucleotide-handling proteins are known in the art and some exemplary polynucleotide-handling proteins are described in more detail below.

In one embodiment, a motor protein is or is derived from a polynucleotide handling enzyme. A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position.

In some embodiments, a polynucleotide-handling protein can be present on the analyte prior to its contact with a nanopore. For example, a polynucleotide-handling protein can be present on a polynucleotide analyte, or on an adapter attached to the polynucleotide analyte.

In some embodiments, the polynucleotide-handling protein is modified to prevent the polynucleotide-handling protein disengaging from the polynucleotide or adapter (other than by passing off the end of the polynucleotide/adapter). The polynucleotide-handling protein can be adapted in any suitable way. For example, the polynucleotide-handling protein can be loaded onto the adapter or polynucleotide and then modified in order to prevent it from disengaging. Alternatively, the polynucleotide-handling protein can be modified to prevent it from disengaging before it is loaded onto the adapter or polynucleotide. Modification of a polynucleotide-handling protein in order to prevent it from disengaging from a polynucleotide or adapter can be achieved using methods known in the art, such as those discussed in WO 2014/013260, which is hereby incorporated by reference in its entirety, and with particular reference to passages describing the modification of motor proteins (polynucleotide binding proteins) such as helicases in order to prevent them from disengaging with polynucleotide strands.

For example, the polynucleotide-handling protein may have a polynucleotide-unbinding opening; e.g. a cavity, cleft or void through which a polynucleotide strand may pass when the polynucleotide-handling protein disengages from the strand. In some embodiments, the polynucleotide-unbinding opening for a given motor protein (polynucleotide binding protein) can be determined by reference to its structure, e.g. by reference to its X-ray crystal structure. The X-ray crystal structure may be obtained in the presence and/or the absence of a polynucleotide substrate. In some embodiments, the location of a polynucleotide-unbinding opening in a given polynucleotide-handling protein may be deduced or confirmed by molecular modelling using standard packages known in the art. In some embodiments, the polynucleotide-unbinding opening may be transiently produced by movement of one or more parts e.g. one or more domains of the polynucleotide-handling protein.

The polynucleotide-handling protein may be modified by closing the polynucleotide-unbinding opening. Closing the polynucleotide-unbinding opening may therefore prevent the polynucleotide-handling protein from disengaging from the polynucleotide or adapter. For example, the polynucleotide-handling protein may be modified by covalently closing the polynucleotide-unbinding opening. In some embodiments, a polynucleotide-handling protein for addressing in this way is a helicase, as described herein.

In one embodiment, a polynucleotide-handling protein is or is derived from a polynucleotide handling enzyme. A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position.

In one embodiment, the polynucleotide-handling protein is derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31.

In some embodiments of the claimed methods, the polynucleotide-handling protein is a helicase, a polymerase, an exonuclease, a topoisomerase, an unfoldase, or a variant thereof.

In one embodiment, the polynucleotide-handling protein is an exonuclease. Suitable enzymes include, but are not limited to, exonuclease I from E. coli (SEQ ID NO: 1), exonuclease III enzyme from E. coli (SEQ ID NO: 2), RecJ from T. thermophilus (SEQ ID NO: 3) and bacteriophage lambda exonuclease (SEQ ID NO: 4), TatD exonuclease and variants thereof. Three subunits comprising the sequence shown in SEQ ID NO: 3 or a variant thereof interact to form a trimer exonuclease.

In one embodiment, the polynucleotide-handling protein is a polymerase. The polymerase may be PyroPhage® 3173 DNA Polymerase (which is commercially available from Lucigen® Corporation), SD Polymerase (commercially available from Bioron®), Klenow from NEB or variants thereof. In one embodiment, the enzyme is Phi29 DNA polymerase (SEQ ID NO: 5) or a variant thereof. Modified versions of Phi29 polymerase that may be used in the invention are disclosed in U.S. Pat. No. 5,576,204.

In one embodiment the polynucleotide-handling protein is a topoisomerase. In one embodiment, the topoisomerase is a member of any of the Moiety Classification (EC) groups 5.99.1.2 and 5.99.1.3. The topoisomerase may be a reverse transcriptase, which are enzymes capable of catalysing the formation of cDNA from a RNA template. They are commercially available from, for instance, New England Biolabs® and Invitrogen®.

In one embodiment, the polynucleotide-handling protein is a helicase. Any suitable helicase can be used in accordance with the methods provided herein. For example, the or each polynucleotide-handling protein used in accordance with the present disclosure may be independently selected from a He1308 helicase, a RecD helicase, a TraI helicase, a TrwC helicase, an XPD helicase, and a Dda helicase, or a variant thereof. Monomeric helicases may comprise several domains attached together. For instance, TraI helicases and TraI subgroup helicases may contain two RecD helicase domains, a relaxase domain and a C-terminal domain. The domains typically form a monomeric helicase that is capable of functioning without forming oligomers. Particular examples of suitable helicases include He1308, NS3, Dda, UvrD, Rep, PcrA, PifI and TraI. These helicases typically work on single stranded DNA. Examples of helicases that can move along both strands of a double stranded DNA include FtfK and hexameric enzyme complexes, or multisubunit complexes such as RecBCD.

Hel308 helicases are described in publications such as WO 2013/057495, the entire contents of which are incorporated by reference. RecD helicases are described in publications such as WO 2013/098562, the entire contents of which are incorporated by reference. XPD helicases are described in publications such as WO 2013/098561, the entire contents of which are incorporated by reference. Dda helicases are described in publications such as WO 2015/055981 and WO 2016/055777, the entire contents of each of which are incorporated by reference.

In one embodiment the helicase comprises the sequence shown in SEQ ID NO: 6 (Trwc Cba) or a variant thereof, the sequence shown in SEQ ID NO: 7 (He1308 Mbu) or a variant thereof or the sequence shown in SEQ ID NO: 8 (Dda) or a variant thereof. Variants may differ from the native sequences in any of the ways discussed herein. An example variant of SEQ ID NO: 8 comprises E94C/A360C. A further example variant of SEQ ID NO: 8 comprises E94C/A360C and then (ΔM1)G1G2 (i.e. deletion of M1 and then addition of G1 and G2).

In some embodiments a polynucleotide-handling protein (e.g. a helicase) can control the movement of polynucleotides in at least two active modes of operation (when the polynucleotide-handling protein is provided with all the necessary components to facilitate movement, e.g. fuel and cofactors such as ATP and Mg²⁺ discussed herein) and one inactive mode of operation (when the polynucleotide-handling protein is not provided with the necessary components to facilitate movement).

When provided with all the necessary components to facilitate movement (i.e. in the active modes), the polynucleotide-handling protein (e.g. helicase) moves along the polynucleotide in a 5′ to 3′ or a 3′ to 5′ direction (depending on the polynucleotide-handling protein). In embodiments in which the polynucleotide-handling protein is used to control the movement of a polynucleotide strand with respect to a nanopore, the polynucleotide-handling protein can be used to either move the polynucleotide away from (e.g. out of) the pore (e.g. against an applied force) or the polynucleotide towards (e.g. into) the pore (e.g. with an applied force). For example, when the end of the polynucleotide towards which the polynucleotide-handling protein moves is captured by a pore, the polynucleotide-handling protein works against the direction of the force and pulls the threaded polynucleotide out of the pore (e.g. into the cis chamber). However, when the end away from which the polynucleotide-handling protein moves is captured in the pore, the polynucleotide-handling protein works with the direction of the force and pushes the threaded polynucleotide into the pore (e.g. into the trans chamber).

When the polynucleotide-handling protein (e.g. helicase) is not provided with the necessary components to facilitate movement (i.e. in the inactive mode) it can bind to the polynucleotide and act as a brake slowing the movement of the polynucleotide when it is moved with respect to a nanopore, e.g. by being pulled into the pore by a force. In the inactive mode, it does not matter which end of the polynucleotide is captured, it is the applied force which determines the movement of the polynucleotide with respect to the pore, and the polynucleotide binding protein acts as a brake. When in the inactive mode, the movement control of the polynucleotide by the polynucleotide binding protein can be described in a number of ways including ratcheting, sliding and braking.

A polynucleotide-handling protein typically requires fuel in order to handle the processing of polynucleotides. Fuel is typically free nucleotides or free nucleotide analogues. The free nucleotides may be one or more of, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are usually selected from AMP, TMP, GMP, CMP, UMP, dAMP, dTMP, dGMP or dCMP. The free nucleotides are typically adenosine triphosphate (ATP).

A cofactor for the polynucleotide-handling protein is a factor that allows the polynucleotide-handling protein to function. The cofactor is preferably a divalent metal cation. The divalent metal cation is preferably Mg²⁺, Mn²⁺, Ca²⁺ or Co²⁺. The cofactor is most preferably Mg²⁺.

Motor proteins suitable for controlling the movement of polypeptide analytes are also known in the art. For example, unfoldase enzymes or variants thereof can be used to control the movement of polypeptides with respect to nanopores. Unfoldase enyzmes include AAA+enzymes such as the ClpX enzyme from E. coli.

### Conditions

The methods disclosed herein may be carried out using any apparatus that is suitable for investigating a membrane/pore system in which a pore is inserted into a membrane. The methods may be carried out using any apparatus that is suitable for transmembrane pore sensing. For example, as explained above, the apparatus may comprise a chamber comprising an aqueous solution and a barrier that separates the chamber into two sections. The barrier may have an aperture in which a membrane containing a transmembrane pore is formed. Transmembrane pores are described herein.

The characterisation methods may be carried out using the apparatus described in WO 2008/102120, WO 2010/122293 or WO 00/28312.

The disclosed methods which comprise characterising an analyte such as polynucleotide or polypeptide methods may involve measuring the ion current flow through the pore, typically by measurement of a current. Alternatively, the ion flow through the pore may be measured optically, such as disclosed by Heron et al: J. Am. Chem. Soc. 9 Vol. 131, No. 5, 2009. Therefore the apparatus may also comprise an electrical circuit capable of applying a potential and measuring an electrical signal across the membrane and pore. The characterisation methods may be carried out using a patch clamp or a voltage clamp. The characterisation methods preferably involve the use of a voltage clamp.

The disclosed methods may be carried out on a silicon-based array of wells where each array comprises 128, 256, 512, 1024, 2000, 3000, 4000, 6000, 10000, 12000, 15000 or more wells.

The disclosed methods which comprise characterising an analyte such as polynucleotide or polypeptide may involve the measuring of a current flowing through the pore. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The disclosed methods are typically carried out in the presence of any charge carriers, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl) or caesium chloride (CsCl) is typically used. KCl is preferred. The salt may be an alkaline earth metal salt such as calcium chloride (CaCl₂)). The salt concentration may be at saturation. The salt concentration may be 3M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The characterisation method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of binding/no binding to be identified against the background of normal current fluctuations.

The disclosed methods are typically carried out in the presence of a buffer. In the exemplary apparatus discussed above, the buffer is present in the aqueous solution in the chamber. Any suitable buffer may be used. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The disclosed methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The characterisation methods are typically carried out at room temperature. The characterisation methods are optionally carried out at a temperature that supports enzyme function, such as about 37° C.

The disclosed methods are typically carried out in vitro.

### Further Aspects of the Disclosure

The present disclosure also provides an amphiphilic layer comprising a tethering complex, obtainable by the disclosed methods.

Also provided is an amphiphilic layer comprising a transmembrane nanopore and a tethering complex, wherein the tethering complex comprises a hydrophobic linker spanning the amphiphilic layer and connected to one ore more hydrophilic components. In some embodiments the amphiphilic layer is partitioned into a first region containing the nanopore, and a second region, and the first region differs chemically and/or physically from the second region. In some embodiments the tethering complex comprises a first hydrophilic component connected to a second hydrophilic component by a hydrophobic linker.

Also provided is an amphiphilic layer comprising a transmembrane nanopore and a tethering complex, wherein the tethering complex comprises a hydrophobic linker spanning the amphiphilic layer, a first hydrophilic component located on the cis side of the amphiphilic layer and a second hydrophilic component located on the trans side of the amphiphilic layer.

In some embodiments the amphiphilic layer comprises a first region and a second region; and the nanopore is located in the first region and the tethering complex is concentrated in the first region.

In some embodiments the amphiphilic layer is as described herein. In some embodiments the tethering complex is assembled as described herein.

Also provided herein is an array comprising two or more amphiphilic layers as described herein. In some embodiments the array is adapted for insertion into a sensor device.

Also provided is a device comprising such an array, a means for applying a voltage potential across the amphiphilic layers and a means for detecting electrical charges across the amphiphilic layers. In some embodiments the device optionally further comprises a fluidics system for supplying a sample to the amphiphilic layer.

Also provided is a system comprising
an amphiphilic layer comprising a first region and a second region;
a nanopore; and
a tethering complex comprising one or more hydrophilic components connected by a hydrophobic linker.

In some embodiments the tethering complex comprises a first hydrophilic component connected to a second hydrophilic component by a hydrophobic linker.

In some embodiments, the system further comprises an analyte-handling protein for controlling the movement of an analyte with respect to the nanopore. In some embodiments the analyte is a polynucleotide and the analyte-handling protein is a polynucleotide-handling protein.

In some embodiments the amphiphilic layer is as described herein. In some embodiments the tethering complex is assembled as described herein. In some embodiments the nanopore is as described herein. In some embodiments the analyte-handling protein is as described herein.

Also provided is a kit comprising:
a tethering complex comprising one or more hydrophilic components connected by a hydrophobic linker; or components thereof;
a nanopore; and
an analyte-handling protein.
   In some embodiments the tethering complex comprises a first hydrophilic component connected to a second hydrophilic component by a hydrophobic linker.

In some embodiments, the components of the tethering complex are as described herein. In some embodiments the kit comprises (i) a hydrophobic linker; (ii) first hydrophilic components; and (iii) second hydrophilic components. In some embodiments the first hydrophilic component and second hydrophilic component are configured to react with reactive groups on the hydrophobic linker to form the tethering complex. In other embodiments, the kit comprises a first moiety comprising a first hydrophilic component attached to a first hydrophobic moiety comprising a first reactive group; and a second moiety comprising a second hydrophilic component attached to a second hydrophobic moiety comprising a second reactive group.

In some embodiments the kit further comprises amphiphilic molecules for forming an amphiphilic layer.

In some embodiments the tethering complex or components thereof, the nanopore and the analyte-handling protein, and the amphiphilic molecules if present, are as described herein.

The kit may be configured for use with an algorithm, also provided herein, adapted to be run on a computer system. The algorithm may be adapted to detect information characteristic of an analyte attached to the tethering complex (e.g. characteristic of the sequence of the analyte, when the analyte is a polypeptide or a polynucleotide), and to selectively process the signal obtained as the analyte moves with respect to the nanopore. Also provided is a system comprising computing means configured to detect information characteristic of an analyte attached to the tethering complex (e.g. characteristic of the sequence of the analyte, when the analyte is a polypeptide or a polynucleotide) and to selectively process the signal obtained as the analyte moves with respect to the nanopore. In some embodiments the system comprises receiving means for receiving data from detection of the analyte, processing means for processing the signal obtained as the analyte moves with respect to the nanopore, and output means for outputting the characterisation information thus obtained.

It is to be understood that although particular embodiments, specific configurations as well as materials and/or molecules, have been discussed herein for methods according to the present invention, various changes or modifications in form and detail may be made without departing from the scope and spirit of this invention. The following examples are provided for illustration only, and should not be considered limiting the application. The application is limited only by the claims.

### EXAMPLES

The following non-limiting examples demonstrate the efficacy of the disclosed methods.

### Example 1

Example 1 describes the non-covalent formation of a tethering complex in a tri-block amphiphilic layer.

### Materials and Methods

The following oligonucleotides were purchased from IDTDNA. The sequences and modifications are shown below.

SEQ ID NO: 11
5′-/A1a/TACTTCGTTATTCTTGTCTCTAT/Cy5/-3′
SEQ ID NO: 12
5′-/Cy5/ATAGAGACAAGAATAACGAAGTA/Cy5/-3′

A1a is an attachment group that reacts selectively with A1b to form a stable covalent bond.

The following buffer solutions were produced:

| Buffer | Components |
|---|---|
| A | 25 mM potassium phosphate, 150 mM potassium ferrocyanide, |
| | 50 mM potassium ferricyanide, pH 8.0 |
| B | 0.5 mg/mL A2a (A2a is a large hydrophilic attachment group |
| | that selectively forms a high affinity non-covalent interaction |
| | with A2b) |
| | 25 mM potassium phosphate, 150 mM potassium ferrocyanide, |
| | 150 mM potassium ferricyanide, pH 8.0 |
| C | 1 μM DNA (SEQ ID NO: 12) |
| | 25 mM potassium phosphate, 150 mM potassium ferrocyanide, |
| | 50 mM potassium ferricyanide, pH 8.0 |
| M | 0.2-20 mg/mL pMOXAₙ-pDMSₓ-pMOXAₙ tri-block |
| | (n = 1-20; x = 5-80), at a 99:1 ratio with |
| | A2b-pDMSₓ-pMOXAₙ-[tether-oligo], (where A2b is an attach- |
| | ment group that forms a high-affinity non-covalent interaction |
| | selectively with A2a, n = 1-20 and x = 5-80) in pDMS |
| | silicone oil |

This example used streptavidin as A2a and biotin as A2b.

Synthesis of Tri-Block, and Hydrophobic-Linker (A2b-pDMSₓ-pMOXAₙ-[Tether-Oligo], where A2b is an Attachment Group that Forms a High-Affinity Non-Covalent Interaction Selectively with A2a, where n=1-20 and x=5-80)

Synthesis of pDMS/pMOXA block copolymer molecules is described in WO 2001/032146. A2b-pDMSₓ-pMOXAₙ-[tether-oligo] (where A2b is an attachment group that forms a high-affinity non-covalent interaction selectively with A2a, n=1-20 and x=5-80) was synthesised using synthetic methods described in WO 2001/032146 and standard synthetic methods. SEQ ID NO: 11 was used for attachment of the [tether-oligo]. The precursor containing the hydrophobic linker contained A2b and A1b. The A1b group was used to conjugate the hydrophobic linker to the A1a group on SEQ ID NO:11, to yield the final product: A2b-pDMSx-pMOXAn-[tether-oligo].

### Generating Membrane Solution “M”

Preparation of membrane solutions is described in WO 2014/064444. Oils with hydrophobic-linker were prepared using the methods described in WO 2014/064444. Solution “M” was prepared by dissolving 0.2-20 mg/mL pMOXAₙ-pDMSₓ-pMOXAₙ tri-block (where n=1 to 20 and x=5 to 80), at a 99:1 ratio with A2b-pDMSₓ-pMOXAₙ-[tether-oligo], (where A2b is an attachment group that forms a high-affinity non-covalent interaction selectively with A2a, where n=1-20 and x=5-80) in pDMS silicone oil.

### Assembling a Trans-Anchored Tether-Oligo

MinION flowcells (Oxford Nanopore Technologies) were assembled to form a product with buffer “A” or “B” in the trans-wells, which contact membranes formed from membrane-solution “M”, which contact buffer “A” as the cis-solution.

### Confocal Microscopy Measurements

To increase fluorescence from cis-accessible tether-oligo (SEQ ID NO: 11), the cis-buffer was replaced with Buffer “C”, which contains SEQ ID NO: 12. SEQ ID NO: 12 can hybridise to SEQ ID NO: 11 and thereby increase its fluorescence.

Flowcells were imaged in the presence of Buffer “C”.

Z-stack imaging was performed on assembled flowcells by confocal microscopy using a 10×M plan lens (0.28 NA); excitation was performed using a 640 nm laser, emission was collected using a C2 detector and a Cy5 filter set. Z-stacks were reconstructed into a 3D image using NIS elements software. Images are displayed using a red lookup table.

Membranes were formed on a MinION chips, with or without trans-anchor. After membrane formation, confocal microscopy was used to generate reconstructed 3D images of membrane fluorescence, as shown in FIGS. 1A and 1B, in which.

### Trans Buffer

FIG. 1A: Buffer A (no trans-anchor).

FIG. 1B: 0.5 mg/mL A2a (where A2a is a large hydrophilic attachment group that forms a high-affinity non-covalent interaction selectively with A2b)

### Membrane Solution:

M (1% A2b-pDMSx-pMOXAn-[tether-oligo]),
(where A2b is an attachment group that forms a high affinity non-covalent interaction selectively with A2a, n=1-20 and x=5-80)

### Cis Buffer

C (Fluorescent SEQ ID NO: 12)

### Results

A2a is a large hydrophilic trans-anchor that is able to form a high-affinity non-covalent interaction selectively with A2b on the hydrophobic linker.

In FIG. 1A, control flowcells were assembled in the absence of A2a trans-anchor, and a fluorescent oligonucleotide was added to the cis buffer, to fluorescently label any cis-accessible tether-oligo. Here, we see that the fluorescent label is dispersed homogeneously throughout the cis solution. This suggests that there is no detectable amount of cis-accessible tether-oligo on the membrane.

In FIG. 1B, flowcells were assembled with 0.5 mg/mL A2a trans-anchor. Here, we see fluorescence from the membranes. This suggests that the inclusion of A2a has increased the availability of tether-oligo at the membranes, and that SEQ ID NO: 11 is able to bind to SEQ ID NO: 12. This is consistent with successful assembly of a trans-anchored tether-oligo, and subsequent accumulation of tether at the membranes.

### Example 2

Example 2 describes the non-covalent formation of a tethering complex in a tri-block amphiphilic layer.

### Materials and Methods

The following oligonucleotides were purchased from IDTDNA or ADTBio. The sequences and modifications are shown below.

SEQ ID NO: 12
5′-/Cy5/ATAGAGACAAGAATAACGAAGTA/Cy5/-3′
SEQ ID NO: 13
5′-/A3a//HEG//HEG//HEG/TACTTCGTTATTCTTGTCTCTAT-3′
SEQ ID NO: 14
5′-/A3a/TACTTCGTTATTCTTGTCTCTAT/Cy5/-3′

A3a is a reactive attachment group that reacts selectively with A3b to form a stable covalent bond. HEG=hexaethylene glycol.

The following buffer solutions were produced:

| Buffer | Components |
|---|---|
| A | 25 mM potassium phosphate, 150 mM potassium ferrocyanide, |
| | 50 mM potassium ferricyanide, pH 8.0 |
| C | 1 μM DNA (SEQ ID NO: 12), 25 mM potassium phosphate, |
| | 150 mM potassium ferrocyanide, 50 mM potassium ferri- |
| | cyanide, pH 8.0 |
| D | 25 μM A1a-H1 (where A1a is an attachment group that reacts |
| | selectively with A1b to form a stable covalent bond, and |
| | H1 is a hydrophilic moiety), in 25 mM potassium phosphate, |
| | 150 mM potassium ferrocyanide, 150 mM potassium ferri- |
| | cyanide, pH 8.0 |
| E | 1 μM DNA (SEQ ID NO: 13) in 25 mM potassium phosphate, |
| | 150 mM potassium ferrocyanide, 150 mM potassium ferri- |
| | cyanide, pH 8.0 |
| F | 1 μM DNA (SEQ ID NO: 14) in 25 mM potassium phosphate, |
| | 150 mM potassium ferrocyanide, 150 mM potassium ferri- |
| | cyanide, pH 8.0 |
| P | 0.2-20 mg/mL pMOXAₙ-pDMSₓ di-block (where n = 1-20 and |
| | x = 5-80), mixed in a 999:1 ratio with A1b-pDMSₓ-A3b |
| | (where A1b is an attachment group that reacts selectively |
| | with A1a to form a stable covalent bond, A3b is an attach- |
| | ment group that reacts selectively with A3a to form a stable |
| | covalent bond, and x = 5-80), in pDMS silicone oil |
| Q | 0.2-20 mg/mL pMOXAₙ-pDMSₓ di-block (where n = 1-20 and |
| | x = 5-80), mixed in a 999:1 ratio with A1b-pDMSₓ-A3b |
| | (where A1b is an attachment group that reacts selectively |
| | with A1a to form a stable covalent bond, A3b is an attach- |
| | ment group that reacts selectively with A3a to form a stable |
| | covalent bond, and x = 5-80), in pDMS silicone oil |

Synthesis of Hydrophobic-Linkers (A1b-pDMSₓ-A3b, where A1b is an Attachment Group that Reacts Selectively with A1a to Form a Stable Covalent Bond, A3b is an Attachment Group that Reacts Selectively with A3a to Form a Stable Covalent Bond, and x=5-80)

Synthesis of pDMS/pMOXA block copolymer molecules is described in WO 2001/032146. The number of pDMS units in A1b-pDMSₓ-A3b in Membrane solution “Q” is 4× longer than the number of pDMS units in Membrane solution “P”. The products were synthesised using methods described in WO 2001/032146 and standard synthetic methods. Due to the synthetic route used, the A1b-pDMSₓ-A3b products will contain some undesired symmetric side-products (i.e. A1b-pDMSₓ-A1b and A3b-pDMSₓ-A3b).

### Generating Membrane Solutions “P” and “Q”

Preparation of membrane solutions is described in WO 2014/064444. Oils with hydrophobic-linker were prepared using methods described in WO 2014/064444.

Solution “P” was prepared by dissolving 0.2-20 mg/mL pMOXAₙ-pDMSₓ di-block (where n=1-20 and x=5-80) and A1b-pDMSx-A3b (where x=5-80) in a 999:1 ratio, in pDMS silicone oil.

Solution “Q” was prepared by dissolving 0.2-20 mg/mL pMOXAₙ-pDMSₓ di-block (where n=1-20 and x=5-80) and A1b-pDMSx-A3b (where x=5-80) in a 999:1 ratio, in pDMS silicone oil.

### Assembling a Trans-Anchored Tether-Oligo

MinION flowcells (Oxford Nanopore technologies) were assembled, to form a product with: buffer “A” or “D” in the trans-wells, which contact membranes formed from membrane-solutions “P” or “Q”, which contact buffer “E” or “F” as the cis-solution. After attachment of tether-oligo to the hydrophobic-linker, the cis buffer is replaced with buffer “A” to remove excess unreacted tether-oligo.

### Confocal Microscopy Measurements

To fluorescently label cis-accessible tether-oligo, the cis-buffer was replaced with Buffer “C”. The fluorescent-oligo in Buffer “C” can hybridise to the tether-oligo and thereby increase its fluorescence. Before imaging, the cis-buffer was replaced with Buffer “A” to remove non-hybridised fluorescent-oligo.

Z-stack imaging was performed on assembled flowcells by confocal microscopy using a 10×M plan lens (0.28 NA); excitation was performed using a 640 nm laser, emission was collected using a C2 detector and a Cy5 filter set. Z-stacks were reconstructed into a 3D image using NIS elements software. Images are displayed using a red lookup table.

Membranes were formed on MinION chips, using di-block copolymer membranes.

Trans anchored tether-oligos were assembled from three separate components in three separate compartments (trans-buffer, membrane, and cis-buffer). Assembly of the final trans-tethered complex drives accumulation of tether-oligo onto the membranes.

A1a-H1 is used as the trans-anchor, and is added into the trans buffer. H1 is inhibited from “flip-flopping” across the membrane, and the A1a group is used for attachment onto the A1b group of the hydrophobic-linker.

Tether-oligo is added into the cis buffer. Tether-oligos contain a A3a group, which is used for attachment to the A3b group of the hydrophobic-linker. Tether oligos were made using either DNA or morpholino chemistry.

The hydrophobic-linker is added into the membrane solution. The hydrophobic-linkers have two ends, each of which contains a different, orthogonally reactive group (A1b and A3b). This ensures that the A3b end of the linker can only react with the A3a-oligo (tether) in the cis buffer, and the A1b end can only react with A1a-H1 (anchor) in the trans buffer.

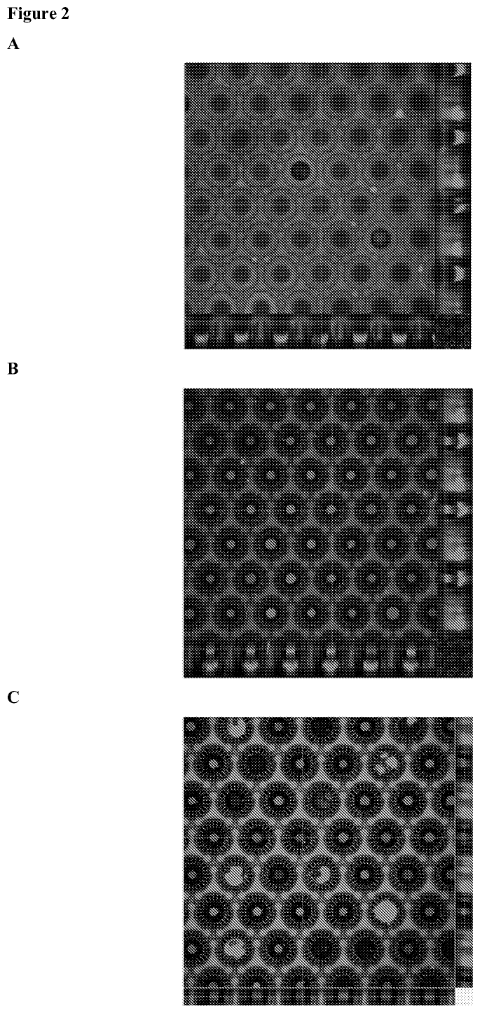

After membrane formation, tether was labelled using solution “C”, and confocal microscopy was used to generate reconstructed 3D images of membrane fluorescence, as shown in FIGS. 2A-C, in which

### FIGS. 2A, 2B

Membrane solution:
Q (A1b-pDMSₓ-A3b, where A1b is an attachment group that reacts selectively with A1a to form a stable covalent bond, A3b is an attachment group that reacts selectively with A3a to form a stable covalent bond, and x=5-80)

### Cis Buffer

F (DNA tether-oligo with A3a reactive group)

### Trans Buffer

FIG. 2A: Trans buffer “A” (no trans-anchor)
FIG. 2B: Trans buffer “D” (A1a-H1 trans-anchor, where A1a is an attachment group that reacts selectively with A1b to form a stable covalent bond, and H1 is a hydrophilic moiety)

### FIG. 2C

### Membrane Solution:

P (A1b-pDMSₓ-A3b, where A1b is an attachment group that reacts selectively with A1a to form a stable covalent bond, A3b is an attachment group that reacts selectively with A3a to form a stable covalent bond, and x=5-80)

### Cis Buffer

E (Morpholino tether-oligo with A3a reactive group)

### Trans Buffer

D (A1a-H1 trans-anchor, where A1a is an attachment group that reacts selectively with A1b to form a stable covalent bond, and H1 is a hydrophilic moiety)

### Results

In FIG. 2A, no trans anchor is added, and in FIG. 2B, A1a-H1 trans-anchor is added. Inclusion of the trans-anchor caused more tether to accumulate on the membranes, demonstrating that attachment of trans-anchor results in accumulation of tether on the membrane.

In FIG. 2B, a longer hydrophobic-linker and a morpholino tether is used. In FIG. 2C, a shorter hydrophobic-linker and a DNA tether is used. The difference in pDMS length is 4-fold. In both cases, fluorescence is observed on the membranes, suggesting that tether-oligo has successfully been accumulated by generation of trans-anchored tether-oligo complexes. This suggests that a wide range of linker lengths and tether chemistries can be used for accumulation of tether-oligo by trans-anchoring.

### Example 3

Example 3 describes the covalent formation of a tethering complex in a tri-block amphiphilic layer.

### Materials and Methods

The following oligonucleotides were purchased from IDTDNA or ADTBio. The sequences and modifications are shown below.

SEQ ID NO: 12
5′-/Cy5/ATAGAGACAAGAATAACGAAGTA/Cy5/-3′
SEQ ID NO: 14
5′-/A3a/TACTTCGTTATTCTTGTCTCTAT/Cy5/-3′

A3a is a reactive attachment group that reacts selectively with A3b to form a stable covalent bond.

The following buffer solutions were produced:

| Buffer | Components |
|---|---|
| A | 25 mM potassium phosphate, 150 mM potassium ferrocyanide, |
| | 50 mM potassium ferricyanide, pH 8.0 |
| C | 1 μM DNA (SEQ ID NO: 12), 25 mM potassium phosphate, |
| | 150 mM potassium ferrocyanide, 50 mM potassium ferri- |
| | cyanide, pH 8.0 |
| F | 1 μM DNA (SEQ ID NO: 14) in 25 mM potassium phosphate, |
| | 150 mM potassium ferrocyanide, 150 mM potassium ferri- |
| | cyanide, pH 8.0 |
| G | 40 μM A1a-H2 (where A1a is an attachment group that reacts |
| | selectively with A1b to form a stable covalent bond, and H2 |
| | is a hydrophilic moiety), in 25 mM potassium phosphate, 150 |
| | mM potassium ferrocyanide, 150 mM potassium ferricyanide, |
| | pH 8.0 |
| R | 0.2-20 mg/mL pDMSₓ-pMOXAₙ-pDMSₓ tri-block (where n = |
| | 1-20 and x = 5-80), mixed in a 99:1 ratio with |
| | A1b-pDMSx-pMOXAₙ-A3b (where A1b is an attachment group |
| | that reacts selectively with A1a to form a stable covalent bond, |
| | A3b is an attachment group that reacts selectively with A3a to |
| | form a stable covalent bond, n = 1-20 and x = 5-80), in pDMS |
| | silicone oil |

Synthesis of Hydrophobic-Linker (A1b-pDMSₓ-pMOXAₙ-A3b, where A1b is an Attachment Group that Reacts Selectively with A1a to Form a Stable Covalent Bond, A3b is an Attachment Group that Reacts Selectively with A3a to Form a Stable Covalent Bond, n=1-20 and x=5-80)

Synthesis of pDMS/pMOXA block copolymer molecules is described in WO 2001/032146. A1b-pDMSₓ-pMOXAₙ-A3b was synthesised using methods described in WO 2001/032146A3 and standard synthetic methods. Due to the synthetic route used, the A1b-pDMSₓ-pMOXAₙ-A3b product will contain some undesired symmetric side-products (i.e. A1b-pDMSₓ-A1b and A3b-pMOXAₙ-pDMSₓ-pMOXAₙ-A3b).

### Generating Membrane Solution “R”

Preparation of membrane solutions is described in WO 2014/064444. Oil with hydrophobic-linker was prepared using methods described in WO 2014/064444. Solution “R” was prepared by dissolving 0.2-20 mg/mL pDMSₓ-pMOXAₙ-pDMSₓ tri-block (where n=1-20 and x=5-80) and A1b-pDMSₓ-pMOXAₙ-A3b (where A1b is an attachment group that reacts selectively with A1a to form a stable covalent bond, A3b is an attachment group that reacts selectively with A3a to form a stable covalent bond, and x=5-80) in a 99:1 ratio, in pDMS silicone oil.

### Assembling a Trans-Anchored Tether-Oligo

MinION flowcells (Oxford Nanopore Technologies) were assembled, to form a product with: buffer “A” or “G” in the trans-wells, which contact membranes formed from membrane-solution “R”, which contact buffer “F” as the cis-solution. After attachment of tether-oligo to the hydrophobic-linker, the cis buffer is replaced with buffer “A” to remove excess unreacted tether-oligo.

### Confocal Microscopy Measurements

To fluorescently label cis-accessible tether-oligo, the cis-buffer was replaced with Buffer “C”. The fluorescent-oligo in Buffer “C” can hybridise to the tether-oligo and thereby increase its fluorescence. Before imaging, the cis-buffer was replaced with Buffer “A” to remove non-hybridised fluorescent-oligo.

Z-stack imaging was performed on assembled flowcells by confocal microscopy using a 10×M plan lens (0.28 NA); excitation was performed using a 640 nm laser, emission was collected using a C2 detector and a Cy5 filter set. Z-stacks were reconstructed into a 3D image using NIS elements software. Images are displayed using a “fire” lookup table.

Membranes were formed on MinION chips, using a tri-block copolymer membrane. Trans anchored tether-oligos were assembled from three separate components in three separate compartments (trans-buffer, membrane, and cis-buffer). Assembly of the final trans-tethered complex drives accumulation of tether-oligo onto the membranes.

A1a-H2 is used as the trans-anchor, and is added into the trans buffer. H2 is inhibited from “flip-flopping” across the membrane, and the A1a group is used for attachment onto the A1b group of the hydrophobic-linker.

Tether-oligo is added into the cis buffer. Tether-oligos contain a A3a group, which is used for attachment to the A3b group of the hydrophobic-linker.

The hydrophobic-linker is added into the membrane solution. The hydrophobic-linkers have two ends, each of which contains a different, orthogonally reactive group (A1b and A3b). This ensures that the A3b end of the linker can only react with the A3a-oligo (tether) in the cis buffer, and the A1b end can only react with A1a-H2 (anchor) in the trans buffer.

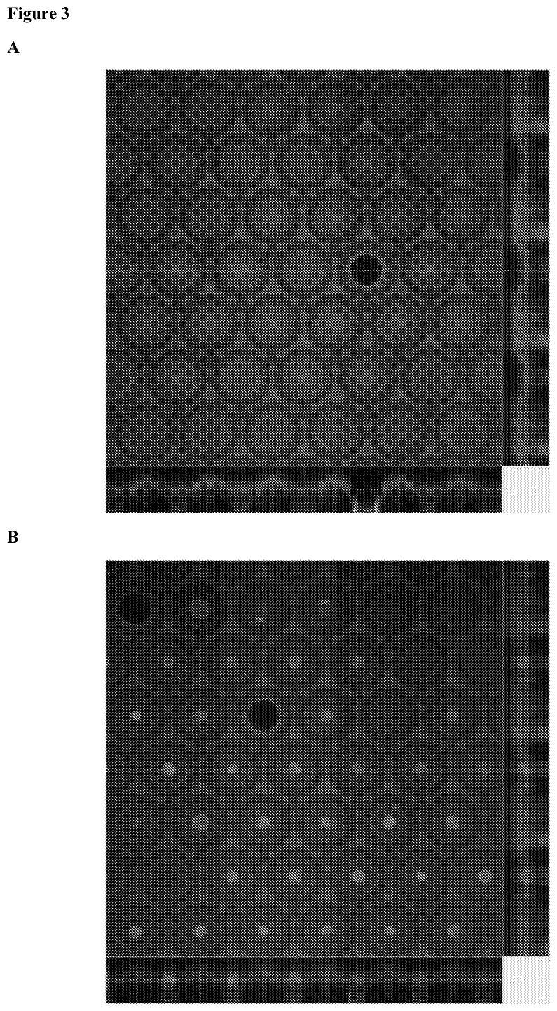

After membrane formation, tether was labelled using solution “C”, and confocal microscopy was used to generate reconstructed 3D images of membrane fluorescence, as shown in FIGS. 3A-B, in which

### Membrane Solution:

R (A1b-pDMSₓ-pMOXAₙ-A3b, where A1b is an attachment group that reacts selectively with A1a to form a stable covalent bond, A3b is an attachment group that reacts selectively with A3a to form a stable covalent bond, n=1-20 and x=5-80)

### Cis Buffer

F (DNA tether-oligo with A3a reactive group)

### Trans Buffer

FIG. 3A: A (no trans-anchor)
FIG. 3B: G (A1a-H2 trans-anchor, where A1a is an attachment group that reacts selectively with A1b to form a stable covalent bond, and H2 is a hydrophilic moiety)

### Results

In FIG. 3A, no trans anchor is added, and in FIG. 3B, A1a-H2 trans-anchor is added. Inclusion of the trans-anchor caused more tether to accumulate on the membranes, demonstrating that attachment of trans-anchor results in accumulation of tether on the membrane.

In this example, the majority of amphiphiles in membrane solution “R” are tri-block molecules, whereas in example 2, the majority of amphiphiles in membrane solutions “P” and “Q” are di-block molecules. In both cases, accumulation of tether was observed upon addition of trans-anchor. This suggests that accumulation of tether can be achieved irrespective of the composition of the membrane.

### Description of the Sequence Listing

SEQ ID NO: 1 shows the amino acid sequence of (hexa-histidine tagged) exonuclease I (EcoExo I) from E. coli.
SEQ ID NO: 2 shows the amino acid sequence of the exonuclease III enzyme from E. coli. SEQ ID NO: 3 shows the amino acid sequence of the RecJ enzyme from T. thermophilus (TthRecJ-cd).
SEQ ID NO: 4 shows the amino acid sequence of bacteriophage lambda exonuclease. The sequence is one of three identical subunits that assemble into a trimer. (http://www.neb.com/nebecomm/products/productM0262.asp).
SEQ ID NO: 5 shows the amino acid sequence of Phi29 DNA polymerase from Bacillus subtilis.
SEQ ID NO: 6 shows the amino acid sequence of Trwc Cba (Citromicrobium bathyomarinum) helicase.
SEQ ID NO: 7 shows the amino acid sequence of He1308 Mbu (Methanococcoides burtonii) helicase.
SEQ ID NO: 8 shows the amino acid sequence of the Dda helicase 1993 from Enterobacteria phage T4.
SEQ ID NO: 11 shows the nucleotide sequence of an oligonucleotide used in the Examples.
SEQ ID NO: 12 shows the nucleotide sequence of an oligonucleotide used in the Examples.
SEQ ID NO: 13 shows the nucleotide sequence of an oligonucleotide used in the Examples.
SEQ ID NO: 14 shows the nucleotide sequence of an oligonucleotide used in the Examples.

SEQUENCE LISTING
exonuclease I from E. coli
SEQ ID NO: 1
MMNDGKQQSTFLFHDYETFGTHPALDRPAQFAAIRTDSEFNVIGEPEVFYCKPADDYLPQ
PGAVLITGITPQEARAKGENEAAFAARIHSLFTVPKTCILGYNNVRFDDEVTRNIFYRNF
YDPYAWSWQHDNSRWDLLDVMRACYALRPEGINWPENDDGLPSFRLEHLTKANGIEHSNA
HDAMADVYATIAMAKLVKTRQPRLFDYLFTHRNKHKLMALIDVPQMKPLVHVSGMFGAWR
GNTSWVAPLAWHPENRNAVIMVDLAGDISPLLELDSDTLRERLYTAKTDLGDNAAVPVKL
VHINKCPVLAQANTLRPEDADRLGINRQHCLDNLKILRENPQVREKVVAIFAEAEPFTPS
DNVDAQLYNGFFSDADRAAMKIVLETEPRNLPALDITFVDKRIEKLLFNYRARNFPGTLD
YAEQQRWLEHRRQVFTPEFLQGYADELQMLVQQYADDKEKVALLKALWQYAEEIVSGSGH
HHHHH
exonuclease III enzyme from E. coli
SEQ ID NO: 2
MKFVSFNINGLRARPHQLEAIVEKHQPDVIGLQETKVHDDMFPLEEVAKLGYNVFYHGQK
GHYGVALLTKETPIAVRRGFPGDDEEAQRRIIMAEIPSLLGNVTVINGYFPQGESRDHP1
KFPAKAQFYQNLQNYLETELKRDNPVLIMGDMNISPTDLDIGIGEENRKRWLRTGKCSFL
PEEREWMDRLMSWGLVDTFRHANPQTADRFSWFDYRSKGFDDNRGLRIDLLLASQPLAEC
CVETGIDYEIRSMEKPSDHAPVWATFRR
RecJ enzyme from T. thermophilus
SEQ ID NO: 3
MFRRKEDLDPPLALLPLKGLREAAALLEEALRQGKRIRVHGDYDADGLTGTAILVRGLAA
LGADVHPFIPHRLEEGYGVLMERVPEHLEASDLFLTVDCGITNHAELRELLENGVEVIVT
DHHTPGKTPPPGLVVHPALTPDLKEKPTGAGVAFLLLWALHERLGLPPPLEYADLAAVGT
IADVAPLWGWNRALVKEGLARIPASSWVGLRLLAEAVGYTGKAVEVAFRIAPRINAASRL
GEAEKALRLLLTDDAAEAQALVGELHRLNARRQTLEEAMLRKLLPQADPEAKAIVLLDPE
GHPGVMGIVASRILEATLRPVFLVAQGKGTVRSLAPISAVEALRSAEDLLLRYGGHKEAA
GFAMDEALFPAFKARVEAYAARFPDPVREVALLDLLPEPGLLPQVFRELALLEPYGEGNP
EPLFL
bacteriophage lambda exonuclease
SEQ ID NO: 4
MTPDIILQRTGIDVRAVEQGDDAWHKLRLGVITASEVHNVIAKPRSGKKWPDMKMSYFHT
LLAEVCTGVAPEVNAKALAWGKQYENDARTLFEFTSGVNVTESPIIYRDESMRTACSPDG
LCSDGNGLELKCPFTSRDFMKFRLGGFEAIKSAYMAQVQYSMWVTRKNAWYFANYDPRMK
REGLHYVVIERDEKYMASFDEIVPEFIEKMDEALAEIGFVFGEQWR
Phi29 DNA polymerase
SEQ ID NO: 5
MKHMPRKMYSCAFETTTKVEDCRVWAYGYMNIEDHSEYKIGNSLDEFMAWVLKVQADLYF
HNLKFDGAFIINWLERNGFKWSADGLPNTYNTIISRMGQWYMIDICLGYKGKRKIHTVIY
DSLKKLPFPVKKIAKDFKLTVLKGDIDYHKERPVGYKITPEEYAYIKNDIQIIAEALLIQ
FKQGLDRMTAGSDSLKGFKDIITTKKFKKVFPTLSLGLDKEVRYAYRGGFTWLNDRFKEK
EIGEGMVFDVNSLYPAQMYSRLLPYGEPIVFEGKYVWDEDYPLHIQHIRCEFELKEGYIP
TIQIKRSRFYKGNEYLKSSGGEIADLWLSNVDLELMKEHYDLYNVEYISGLKFKATTGLF
KDFIDKWTYIKTTSEGAIKQLAKLMLNSLYGKFASNPDVTGKVPYLKENGALGFRLGEEE
TKDPVYTPMGVFITAWARYTTITAAQACYDRIIYCDTDSIHLTGTEIPDVIKDIVDPKKL
GYWAHESTFKRAKYLRQKTYIQDIYMKEVDGKLVEGSPDDYTDIKFSVKCAGMTDKIKKE
VTFENFKVGFSRKMKPKPVQVPGGVVLVDDTFTIKSGGSAWSHPQFEKGGGSGGGSGGSA
WSHPQFEK
Trwc Cba helicase
SEQ ID NO: 6
MLSVANVRSPSAAASYFASDNYYASADADRSGQWIGDGAKRLGLEGKVEARAFDALLRGE
LPDGSSVGNPGQAHRPGTDLTFSVPKSWSLLALVGKDERIIAAYREAVVEALHWAEKNAA
ETRVVEKGMVVTQATGNLAIGLFQHDTNRNQEPNLHFHAVIANVTQGKDGKWRTLKNDRL
WQLNTTLNSIAMARFRVAVEKLGYEPGPVLKHGNFEARGISREQVMAFSTRRKEVLEARR
GPGLDAGRIAALDTRASKEGIEDRATLSKQWSEAAQSIGLDLKPLVDRARTKALGQGMEA
TRIGSLVERGRAWLSRFAAHVRGDPADPLVPPSVLKQDRQTIAAAQAVASAVRHLSQREA
AFERTALYKAALDFGLPTTIADVEKRTRALVRSGDLIAGKGEHKGWLASRDAVVTEQRIL
SEVAAGKGDSSPAITPQKAAASVQAAALTGQGFRLNEGQLAAARLILISKDRTIAVQGIA
GAGKSSVLKPVAEVLRDEGHPVIGLAIQNTLVQMLERDTGIGSQTLARFLGGWNKLLDDP
GNVALRAEAQASLKDHVLVLDEASMVSNEDKEKLVRLANLAGVHRLVLIGDRKQLGAVDA
GKPFALLQRAGIARAEMATNLRARDPVVREAQAAAQAGDVRKALRHLKSHTVEARGDGAQ
VAAETWLALDKETRARTSIYASGRAIRSAVNAAVQQGLLASREIGPAKMKLEVLDRVNTT
REELRHLPAYRAGRVLEVSRKQQALGLFIGEYRVIGQDRKGKLVEVEDKRGKRFRFDPAR
IRAGKGDDNLTLLEPRKLEIHEGDRIRWTRNDHRRGLFNADQARVVEIANGKVTFETSKG
DLVELKKDDPMLKRIDLAYALNVHMAQGLTSDRGIAVMDSRERNLSNQKTFLVTVTRLRD
HLTLVVDSADKLGAAVARNKGEKASAIEVTGSVKPTATKGSGVDQPKSVEANKAEKELTR
SKSKTLDFGI
He1308 Mbu helicase
SEQ ID NO: 7
MMIRELDIPRDIIGFYEDSGIKELYPPQAEAIEMGLLEKKNLLAAIPTASGKTLLAELAM
IKAIREGGKALYIVPLRALASEKFERFKELAPFGIKVGISTGDLDSRADWLGVNDIIVAT
SEKTDSLLRNGTSWMDEITTVVVDEIHLLDSKNRGPTLEVTITKLMRLNPDVQVVALSAT
VGNAREMADWLGAALVLSEWRPTDLHEGVLFGDAINFPGSQKKIDRLEKDDAVNLVLDTI
KAEGQCLVFESSRRNCAGFAKTASSKVAKILDNDIMIKLAGIAEEVESTGETDTAIVLAN
CIRKGVAFHHAGLNSNHRKLVENGFRQNLIKVISSTPTLAAGLNLPARRVIIRSYRRFDS
NFGMQPIPVLEYKQMAGRAGRPHLDPYGESVLLAKTYDEFAQLMENYVEADAEDIWSKLG
TENALRTHVLSTIVNGFASTRQELFDFFGATFFAYQQDKWMLEEVINDCLEFLIDKAMVS
ETEDIEDASKLFLRGTRLGSLVSMLYIDPLSGSKIVDGFKDIGKSTGGNMGSLEDDKGDD
ITVTDMTLLHLVCSTPDMRQLYLRNTDYTIVNEYIVAHSDEFHEIPDKLKETDYEWFMGE
VKTAMLLEEWVTEVSAEDITRHFNVGEGDIHALADTSEWLMHAAAKLAELLGVEYSSHAY
SLEKRIRYGSGLDLMELVGIRGVGRVRARKLYNAGFVSVAKLKGADISVLSKLVGPKVAY
NILSGIGVRVNDKHFNSAPISSNTLDTLLDKNQKTFNDFQ
Dda helicase
SEQ ID NO: 8
MTFDDLTEGQKNAFNIVMKAIKEKKHHVTINGPAGTGKTTLTKFIIEALISTGETGIILA
APTHAAKKILSKLSGKEASTIHSILKINPVTYEENVLFEQKEVPDLAKCRVLICDEVSMY
DRKLFKILLSTIPPWCTIIGIGDNKQIRPVDPGENTAYISPFFTHKDFYQCELTEVKRSN
APIIDVATDVRNGKWIYDKVVDGHGVRGFTGDTALRDFMVNYFSIVKSLDDLFENRVMAF
TNKSVDKLNSIIRKKIFETDKDFIVGEIIVMQEPLFKTYKIDGKPVSEIIFNNGQLVRII
EAEYTSTFVKARGVPGEYLIRHWDLTVETYGDDEYYREKIKIISSDEELYKFNLFLGKTA
ETYKNWNKGGKAPWSDFWDAKSQFSKVKALPASTFHKAQGMSVDRAFIYTPCIHYADVEL
AQQLLYVGVTRGRYDVFYV
SEQ ID NO: 11
5′-/A1a/TACTTCGTTATTCTTGTCTCTAT/Cy5/-3′
SEQ ID NO: 12
5′-/Cy5/ATAGAGACAAGAATAACGAAGTA/Cy5/-3′
SEQ ID NO: 13
5′-/A3a//HEG//HEG//HEG/TACTTCGTTATTCTTGTCTCTAT-3
SEQ ID NO: 14
5′-/A3a/TACTTCGTTATTCTTGTCTCTAT/Cy5/-3′

1. A method of concentrating a tethering complex in a region of an amphiphilic layer, said amphiphilic layer comprising a plurality of amphiphilic molecules and a detector, wherein the tethering complex comprises one or more hydrophilic components connected by a hydrophobic linker;
the method comprising contacting the tethering complex or one or more components thereof with said plurality of amphiphilic molecules;
and wherein the amphiphilic layer comprises a first region comprising the detector, and a second region, wherein the first region differs chemically and/or physically from the second region, and wherein the tethering complex preferentially localises to the first region relative to the second region; thereby concentrating the tethering complex in the first region of the amphiphilic layer.

2. A method according to claim 1, wherein the tethering complex comprises a first hydrophilic component connected to a second hydrophilic component by a hydrophobic linker.

3. A method according to claim 1 or claim 2, wherein the first region is a multi-layered region of the amphiphilic layer.

4. A method according to any one of the preceding claims, wherein the first region and the second region both comprise the same type of amphiphilic molecules.

5. A method according to any one of the preceding claims, wherein the first region comprises a first composition of amphiphilic molecules and the second region comprises a second composition of amphiphilic molecules, and the first composition differs from the second composition.

6. A method according to any one of the preceding claims, wherein the first region and the second region of the amphiphilic layer respectively correspond to first and second areas of a substrate, wherein the first area of the substrate differs chemically and/or physically from the second area.

7. A method according to claim 6, wherein the first area corresponds to an aperture in a substrate and the second area corresponds to an optionally coated portion of the substrate.

8. A method according to any one of claims 1 to 5, wherein the first region corresponds to the interfacial surface area between a first droplet and a second droplet pair, wherein the first and second droplets each have an amphiphilic coating; and the second region corresponds to the surface area of the portion of the first droplet which does not interface with a second droplet.

9. A method according to any one of the preceding claims, wherein the first region and the second region are phase separated regions of the amphiphilic layer.

10. A method for assembling a tethering complex in an amphiphilic layer, wherein the tethering complex comprises one or more hydrophilic components connected by a hydrophobic linker; the method comprising contacting the tethering complex or one or more components thereof with a plurality of amphiphilic molecules and subsequently forming the amphiphilic layer.

11. A method for assembling a tethering complex in an amphiphilic layer, wherein the tethering complex comprises one or more hydrophilic components connected by a hydrophobic linker; the method comprising (i) forming the amphiphilic layer from a plurality of amphiphilic molecules; and (ii) contacting the amphiphilic layer with the tethering complex or one or more components thereof

12. A method according to claim 10 or claim 11, comprising (i) contacting the hydrophobic linker with the amphiphilic molecules or the amphiphilic layer; wherein the hydrophobic linker is not attached to at least one of the one or more hydrophilic components when the hydrophobic linker is contacted with the amphiphilic molecules or amphiphilic layer and (ii) attaching at least one of the one or more hydrophilic components to the hydrophobic linker once the amphiphilic layer has been formed, thereby forming the tethering complex.

13. A method for assembling a tethering complex in an amphiphilic layer, wherein the tethering complex comprises a first hydrophilic component connected to a second hydrophilic component by a hydrophobic linker, the method comprising contacting the tethering complex or one or more components thereof with a plurality of amphiphilic molecules and subsequently forming the amphiphilic layer.

14. A method according to claim 13, comprising (i) contacting the hydrophobic linker with the plurality of amphiphilic molecules; and (ii) forming the amphiphilic layer.

15. A method according to claim 14, wherein the hydrophobic linker is attached to at least one of the first hydrophilic component and/or the second hydrophilic component.

16. A method according to any one of claims 13 to 15, wherein the hydrophobic linker is not attached to the first hydrophilic component and/or the second hydrophilic component when the hydrophobic linker is contacted with the amphiphilic molecules, and wherein the method further comprises attaching the first hydrophilic component and/or the second hydrophilic component to the hydrophobic linker once the amphiphilic layer has been formed thereby forming the tethering complex.

17. A method according to any one of claims 13 to 16, comprising providing a mixture comprising amphiphilic molecules and the hydrophobic linker; and
A:
(a) contacting an aperture with the mixture, wherein a buffer comprising the second hydrophilic component is present on the trans side of the aperture, such that an amphiphilic layer comprising the hydrophobic linker forms across the aperture, and the second hydrophilic component attaches to the hydrophobic linker; and
(b) adding a buffer comprising the first hydrophilic component to the cis side of the amphiphilic layer such that the first hydrophilic component attaches to the hydrophobic linker;
or
B:
(a) contacting an aperture with the mixture, wherein a buffer comprising the first hydrophilic component is present on the cis side of the aperture, such that an amphiphilic layer comprising the hydrophobic linker forms across the aperture, and the first hydrophilic component attaches to the hydrophobic linker; and
(b) adding a buffer comprising the second hydrophilic component to the trans side of the amphiphilic layer such that the second hydrophilic component attaches to the hydrophobic linker.

18. A method according to any one of claims 13 to 16, comprising:
(a) providing a mixture comprising amphiphilic molecules and the hydrophobic linker bound first to a first hydrophilic component; and
(b) contacting an aperture with the mixture, wherein a buffer comprising a second hydrophilic component is present on the trans side of the aperture, such that an amphiphilic layer comprising the hydrophobic linker forms across the aperture, and the second hydrophilic component attaches to the hydrophobic linker on the trans side of the membrane.

19. A method according to any one of claims 13 to 16, comprising:
(a) providing a mixture comprising amphiphilic molecules and the hydrophobic linker bound first to a second hydrophilic component; and
(b) contacting an aperture with the mixture, wherein a buffer comprising a first hydrophilic component is present on the cis side of the aperture, such that an amphiphilic layer comprising the hydrophobic linker forms across the aperture, and the first hydrophilic component attaches to the hydrophobic linker on the cis side of the membrane

20. A method for assembling a tethering complex in an amphiphilic layer, wherein the tethering complex comprises a first hydrophilic component connected to a second hydrophilic component by a hydrophobic linker, the method comprising (i) forming the amphiphilic layer from a plurality of amphiphilic molecules; and (ii) contacting the amphiphilic layer with the hydrophobic linker;
wherein the hydrophobic linker is optionally attached to either the first hydrophilic component or the second hydrophilic component.

21. A method according to claim 20, further comprising attaching the first hydrophilic component and/or the second hydrophilic component to the hydrophobic linker once the amphiphilic layer has been formed thereby forming the tethering complex.

22. A method according to any one of claims 13 to 21, comprising contacting the first hydrophilic component; the second hydrophilic component; and the hydrophobic linker; wherein the first hydrophilic component comprises a first reactive group; the second hydrophilic component comprises a second reactive group; and the hydrophobic linker comprises reactive groups; and
reacting the first reactive group with a reactive group on the hydrophobic linker and reacting the second reactive group with a reactive group on the hydrophobic linker so as to connect the first hydrophilic component to the second hydrophilic component by the hydrophobic linker, thereby forming the tethering complex.

23. A method for assembling a tethering complex in an amphiphilic layer, wherein the tethering complex comprises a first hydrophilic component connected to a second hydrophilic component by a hydrophobic linker, the method comprising:
(a) contacting a first moiety with a second moiety, wherein the first moiety comprises the first hydrophilic component attached to a first hydrophobic moiety comprising a first reactive group and the second moiety comprises the second hydrophilic component attached to a second hydrophobic moiety comprising a second reactive group; and
(b) reacting the first reactive group with the second reactive group thereby forming a hydrophobic linker connecting the first hydrophilic component to the second hydrophilic component, thereby forming the tethering complex.

24. A method according to claim 23, wherein the first hydrophilic component is provided from a first face of the amphiphilic layer and the second hydrophilic component is provided from a second face of the amphiphilic layer.

25. A method according to any one of claims 10 to 24, further comprising inserting a detector into the amphiphilic layer.

26. A method according to any one of claims 1 to 9, wherein the tethering complex is assembled according to any one of claims 10 to 24.

27. A method according to any one of the preceding claims, wherein the hydrophobic linker covalently attaches to (i) the one or more hydrophilic components or (ii) the first hydrophilic component and/or the second hydrophilic component.

28. A method according to any one of claims 1 to 27, wherein the hydrophobic linker non-covalently attaches to (i) the one or more hydrophilic components or (ii) the first hydrophilic component and/or the second hydrophilic component.

29. A method according to any one of claims 1 to 27, wherein (i) the hydrophobic linker covalently attaches to the first hydrophilic component and non-covalently attaches to the second hydrophilic component; or (ii) the hydrophobic linker non-covalently attaches to the first hydrophilic component and covalently attaches to the second hydrophilic component.

30. A method according to any one of the preceding claims, wherein the hydrophobic linker comprises or consists of a saturated or non-saturated hydrocarbon or organic molecule or a saturated or non-saturated inorganic molecule;
wherein optionally the hydrophobic linker comprises or consists of a hydrophobic polypeptide, a spiroketal, polydimethylsiloxane (PDMS), an alkane, a protein, a transmembrane pore, a carbon nanotube, a natural lipid or a synthetic lipid-like molecule.

31. A method according to any one of the preceding claims, wherein (i) at least one of the one or more hydrophilic components; or (ii) the first hydrophilic component comprises an analyte-binding moiety;
optionally wherein (i) the analyte binding moiety comprises biotin and the first hydrophilic component comprises streptavidin; (ii) the analyte binding moiety comprises cholesterol and the first hydrophilic component comprises cyclodextrin; or (iii) the first hydrophilic component comprises a nucleotide or polynucleotide.

32. A method according to any one of claims 2 to 31, wherein the second hydrophilic component comprises an anchor or anchor-binding moiety;
optionally wherein (i) the anchor binding moiety comprises biotin and the anchor comprises streptavidin; or (ii) the anchor binding moiety comprises cholesterol and the anchor comprises cyclodextrin; or (iii) the anchor comprises a nucleotide or polynucleotide.

33. A method of concentrating an analyte in the region of a detector, the method comprising:
carrying out the method of any one of claims 1 to 9 or 26 to 32; and
contacting the analyte with the tethering complex such that the analyte attaches to the first hydrophilic component of the tethering complex;
thereby concentrating the analyte in the region of the detector.

34. A method according to claim 33, wherein the analyte binds to a plurality of tethering complexes, thereby concentrating the analyte in the region of the detector.

35. A method of concentrating an analyte in the region of a amphiphilic layer comprising a detector, the method comprising concentrating a plurality of tethering complexes in the region of the detector; and
i) contacting the analyte with said tethering complexes such that the analyte binds to a plurality of said tethering complexes; or
ii) contacting (A) a splint comprising (i) a plurality of binding sites for said tethering complexes and (ii) one or more binding sites for said analyte; and (B) the analyte with said tethering complexes such that the splint binds to a plurality of said tethering complexes and the analyte binds to the splint;
thereby concentrating the analyte in the region of the detector.

36. A method according to claim 35 wherein the tethering complexes and/or the amphiphilic layer are as defined in any one of claims 1 to 32.

37. A method of characterising a target analyte; the method comprising concentrating the analyte in the region of a detector using the method of any one of claims 33 to 36, and taking one or more measurements as the analyte moves with respect to the detector, wherein the one or more measurements are indicative of one or more characteristics of the analyte, and thereby characterising the analyte as it moves with respect to the detector.

38. The method of claim 37, wherein multiple target analytes are characterised.

39. A method according to claim 37 or claim 38, wherein the or each analyte is a polynucleotide, protein, peptide, carbohydrate or metabolite.

40. A method according to any one of claims 1 to 9, 25 or 33 to 39 wherein the detector comprises a transmembrane nanopore capable of characterising an analyte as the analyte moves with respect to the nanopore.

41. An amphiphilic layer obtainable by a method according to any one of claims 1 to 32.

42. An amphiphilic layer comprising a transmembrane nanopore and a tethering complex, wherein the tethering complex comprises a hydrophobic linker spanning the amphiphilic layer, a first hydrophilic component located on the cis side of the amphiphilic layer and a second hydrophilic component located on the trans side of the amphiphilic layer.

43. An amphiphilic layer according to claim 42, comprising a first region and a second region; wherein the first region differs chemically and/or physically from the second region, and wherein the nanopore is located in the first region and the tethering complex is concentrated in the first region.

44. An amphiphilic layer according to claim 42 or claim 43, wherein:
the amphiphilic layer is as defined in any one of claims 3 to 9; and/or
the tethering complex is assembled according to any one of claims 10 to 25 or is as defined in any one of claims 27 to 32.

45. An array comprising two or more amphiphilic layers as defined in any one of claims 41 to 44.

46. A device comprising the array of claim 45, a means for applying a voltage potential across the amphiphilic layers and a means for detecting electrical charges across the amphiphilic layers;
wherein said device optionally further comprises a fluidics system for supplying a sample to the amphiphilic layer.


SEQUENCE LISTING (14 sequences)

SEQ ID NO: 1 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 2 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 3 - protein, 425 aa, Thermus thermophilus
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 4 - protein, 226 aa, Bacteriophage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 5 - protein, 608 aa, Bacillus subtilis phage Phi 29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 6 - protein, 970 aa, Citromicrobium bathyomarinum
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 7 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 8 - protein, 439 aa, Enterobacteria phage T4
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 9-10 - skipped (2 entries)

SEQ ID NO: 11 - DNA, 23 bp, Artificial Sequence
  Synthetic Oligonucleotide
  misc_feature (1)..(1): A1a is an attachment group that reacts selectively with A1b to form a stable covalent bond.
  misc_feature (23)..(23): Cy5 Fluorophore
tacttcgttattcttgtctctat

SEQ ID NO: 12 - DNA, 23 bp, Artificial Sequence
  Synthetic Oligonucleotide
  misc_feature (1)..(1): Cy5 Fluorophore
  misc_feature (23)..(23): Cy5 Fluorophore
atagagacaagaataacgaagta

SEQ ID NO: 13 - DNA, 23 bp, Artificial Sequence
  Synthetic Oligonucleotide
  misc_feature (1)..(1): A3a is a reactive attachment group that reacts selectively with A3b to form a stable covalent bond.
  misc_feature (1)..(1): 3x Hexaethylene glycol units
tacttcgttattcttgtctctat

SEQ ID NO: 14 - DNA, 23 bp, Artificial Sequence
  Synthetic Oligonucleotide
  misc_feature (1)..(1): A3a is a reactive attachment group that reacts selectively with A3b to form a stable covalent bond.
  misc_feature (23)..(23): Cy5 Fluorophore
tacttcgttattcttgtctctat